(19) United States
(12) Patent Application Publication
(10) Pub. No.: US 2023/0235284 A1
(43) Pub. Date: Jul. 27, 2023

(54) SYSTEMS AND METHODS TO MODEL ADAPTIVE IMMUNE RESPONSES

(71) Applicant: The Board of Trustees of the Leland Stanford Junior University, Stanford, CA (US)

(72) Inventors: Mark M. Davis, Atherton, CA (US); Lisa E. Wagar, Santa Ana, CA (US)

(21) Appl. No.: 18/094,851

(22) Filed: Jan. 9, 2023

Related U.S. Application Data
(60) Provisional application No. 63/298,079, filed on Jan. 10, 2022.

(51) Int. Cl. C12N 5/0784; C12N 5/0783; C12N 5/0781

(52) U.S. Cl. CPC C12N 5/0639; C12N 5/0636; C12N 5/0635; A61K 2039/5154

(57) ABSTRACT

Disclosed herein are methods, systems and devices to model adaptive immune responses and develop and/or test improved antibodies, vaccines, and other therapeutic agents. The adaptive immune responses can be modeled using lymphoid tissue derived from a subject.

### CROSS-REFERENCE

This application claims the benefit of U.S. Provisional Patent Application No. 63/298,079, filed Jan. 10, 2022, which application is incorporated herein by reference in its entirety.

### STATEMENT AS TO FEDERALLY SPONSORED RESEARCH

This invention was made with Government support under contracts AI057229 and AI090019 awarded by the National Institutes of Health. The Government has certain rights in the invention.

### BACKGROUND OF THE INVENTION

Antigen recognition by lymphocytes has been studied by immunologists since the discovery of antibodies and their specificities over a century ago, followed by the more recent discovery of T cells and their antigen receptors in the 1960s-1980s. The B cells that are responsible for forming a neutralizing antibody response develop with germinal centers (GCs) and extrafollicular regions in lymphoid organs. Upon antigen presentation by antigen-presenting cells (APCs), T follicular helper (TFH) cells, and a variety of hematopoietic and non-hematopoietic cells interact and deliver signals to GC B cells for survival, proliferation, antibody affinity maturation, class-switch recombination, and differentiations. Almost all of these interactions have been elucidated through in vivo studies of inbred mice. While these have produced a wealth of important information, the lack of a system that replicates the essential features of adaptive immunity, such as affinity maturation and class switching, and the effects of adjuvants, leaves many mechanistic aspects inaccessible.

Much of what is known about adaptive immunity has come from inbred mouse studies, using methods that are often difficult or impossible to confirm in humans. Often vaccine responses in mice are poorly predictive of responses to those same vaccines in humans. The present invention uses cells from lymphoid organs to develop a functional organotypic system that recapitulates key features germinal centers and adaptive immunity in vitro, including but not limited to the production of antigen-specific antibodies, somatic hypermutations, affinity maturation, plasmablast differentiation, and class-switch recombination.

### SUMMARY OF THE INVENTION

Disclosed herein are methods, systems, and devices capable of modeling adaptive immune responses. Disclosed herein is an in-vitro cell cluster comprising lymphoid cells, wherein said in-vitro cell cluster comprises a germinal center and an aggregate of T-cells; wherein said in-vitro cell cluster is configured to maintain said germinal center and said aggregate of T-cells and a cellular respiration for at least 24 hours. In some embodiments, the functions of this cluster can be modulated by one or more adjuvants. In some embodiments, the adjuvants can comprise aluminum hydroxide or imiquimod, or any of adjuvants in current use or those in development. In some embodiments, the germinal center can be configured to perform one or more of: hypermutation maturation, affinity maturation, plasmablast differentiation, class switching recombination, and antigen-specific antibody production. In some embodiments, the cells can be configured to differentiate to form said in-vitro cell cluster upon exposure to an antigen. In some embodiments, the antigen can be a peptide, protein or fragment thereof, polysaccharides, lipids, nucleic acids, or other biomolecules. In some embodiments, the protein can be a viral protein, a growth factor, a cancer related protein, or an auto-immune disease related protein. In some embodiments, the carbohydrate can be a bacterial coat protein or a fragment thereof. Many natural protein antigens, such as influenza hemagglutinin, are glycosylated, and can stimulate protein/peptide specific antibody expressing B cells, or T cells, or these specific lymphocytes can be stimulated by carbohydrates, or glycosylated proteins or peptides in any combination. In some embodiments, the lymphoid cells can be derived from tonsil tissue, spleen tissue, adenoid tissue, thymus tissue, or lymph node tissue. In some embodiments, the germinal center can comprise antigen presenting cells (APCs) and T-cells at least partially surrounding said functional germinal center. In some embodiments, the APCs can comprise B-cells or dendritic cells. In some embodiments, the dendritic cells can comprise follicular dendritic cells. In some embodiments, the B-cells can comprise CD38+ B-cells. In some embodiments, the B-cells can comprise CD27+ B-cells. In some embodiments, the T-cells can comprise CD8+ T-cells or CD4+ T-cells of the αβ type. In some embodiments, the T-cells can comprise γδ T cells.

Disclosed herein is an in-vitro lymphoid culture system comprising a well, the well comprising a cell-suspension of lymphoid cells comprising T-cells, B-cells, and non lymphoid cells that are found in lymphoid organoids and are essential for these reactions (Wagar et al 2021) plus media; wherein said media provides nutrients and factors that are needed for proper cell differentiation and the spatial organization of germinal centers and the adjacent aggregate of T-cells. In some embodiments, the media can comprise recombinant human B-cell activating factor (BAFF) and IL-2. In some embodiments, the media can further comprise one or more adjuvants and a vaccine or vaccine candidate. In some embodiments, the one or more adjuvants can comprise aluminum hydroxide or imiquimod, or many other adjuvants in use or being developed. In some embodiments, the germinal center can perform one or more of: hypermutation maturation, affinity maturation, plasmablast differentiation, class switching recombination, and antigen-specific antibody production. In some embodiments, the cells can be modified by introduction of an antigen to said media. In some embodiments, the antigen can be a protein or fragment thereof, or a carbohydrate or a glycoprotein or fragment thereof. In some embodiments, the protein can be a viral protein, a growth factor, a cancer related protein, or an auto-immune disease related protein. In some embodiments, the lymphoid cells can be derived from one or more of tonsil tissue, spleen tissue, adenoid tissue, thymus tissue, or lymph node tissue. In some embodiments, the germinal center can comprise antigen-presenting cells (APCs). In some embodiments, the aggregate of T-cells is outside of said functional germinal center. In some embodiments, the APCs can comprise B-cells or dendritic cells. In some embodiments, the dendritic cells can comprise follicular dendritic cells. In some embodiments, the B-cells can comprise CD38+ B-cells. In some embodiments, the B-cells can comprise CD27+ B-cells. In some embodiments, the T-cells can comprise CD8+ T-cells or CD4+ T-cells of the αβ type. In some embodiments, the T-cells can comprise γδ T cells.

In some embodiments, the spatial organization of lymphoid tissue can be maintained for at least four days. In some embodiments, the spatial organization of lymphoid tissue can be maintained for at least one week. In some embodiments, the spatial organization of lymphoid tissue can be maintained for at least two weeks. In some embodiments, the spatial organization of lymphoid tissue can be maintained for at least three weeks.

Disclosed herein is a method for generating antibodies from a lymphoid organoid, comprising (a) placing lymphoid cells in a media to produce said lymphoid organoid, wherein said lymphoid organoid comprises a spatial organization of lymphoid tissue, said spatial organization comprising a germinal center and an aggregate of T-cells; (b) introducing an antigen to said media; (c) incubating said lymphoid organoid with said antigen to generate said antibodies; and isolating said antibodies from said lymphoid organoid. In some embodiments, step (a) can further comprise introducing a B-cell activating factor to said media. In some embodiments, step (b) can further comprise introducing an adjuvant to said media. In some embodiments, the adjuvant can be aluminum hydroxide or imiquimod. In some embodiments, step (c) can further comprise incubating said lymphoid organoid with said antigen and said adjuvant. In some embodiments, the adjuvant can be introduced after said antigen. In some embodiments, the incubating can be for at least 48 hours. In some embodiments, the method can further comprise obtaining said lymphoid cells from a subject. In some embodiments, the lymphoid cells can be obtained by biopsy. In some embodiments, the lymphoid cells can be derived from tonsil tissue, spleen tissue, adenoid tissue, thymus tissue, or lymph node tissue. In some embodiments, the antigen can be a peptide, protein or fragment thereof. In some embodiments, the protein is a viral protein, a growth factor, a cancer related protein, or an auto-immune disease related protein. In some embodiments, the virus can be a coronavirus.

Disclosed herein is a method of screening a patient for immune response to a treatment comprising obtaining a sample comprising lymphoid cells from a patient; placing said lymphoid cells in a system as described herein; exposing said lymphoid cells to a treatment to stimulate the production of antibodies; isolating said antibodies from said system; and assaying said antibodies. In some embodiments, the sample can be selected from one or more of tonsil tissue, spleen tissue, adenoid tissue, thymus tissue, or lymph node tissue. In some embodiments, the sample can be obtained by biopsy.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1F show the adaptive immune responses in tonsil organoids. FIG. 1A shows a workflow for tissue disruption and culture preparation with representative stereoscope images from day-5 unstimulated or live attenuated influenza vaccine (LAIV)-stimulated replicate cultures. Light regions are areas of high cell density. FIG. 1B shows cell composition of immune cell types in freshly isolated, frozen and revived day-0 tonsil cells, day-7 unstimulated cultures and day-7 LAIV-stimulated cultures. Frequencies were determined by flow cytometry, and plotted values are the mean±s.e.m. (n=5 donors). PB/PCs, plasmablast/plasma cells; NK, natural killer. FIG. 1C shows Plasmablast differentiation and specific antibody secretion from unstimulated and LAIV-stimulated organoids from day-7 cultures—paired, two-sided Wilcoxon signed-rank tests were used to calculate P values (n=15 donors). OD, optical density. FIG. 1D shows antigen-specific antibodies and total IgG from day-7 unstimulated and LAIV-stimulated organoid cultures. Each symbol represents individual donors (n=6). FIG. 1E shows ASCs specific for influenza antigens from day-7 unstimulated and LAIV-stimulated cultures (n=5 donors). FIG. 1F shows Plasmablast frequencies and antibody secretion on day 14 from immune organoids derived from human lung-draining lymph nodes and spleens (n=2 for each tissue). A/Cal, A/California H1N1 virus.

FIGS. 2A-2F show longitudinal tracking of immune organoids to reveal cellular and functional changes consistent with an adaptive response. FIG. 2A shows representative flow cytometry staining of B cell differentiation phenotypes in unstimulated and LAIV-stimulated cultures—cells shown are pre-gated on total B cells (CD45+CD19+CD3−). FIGS. 2B-2C show quantitation of B cell (FIG. 2B) and T cell (FIG. 2C) phenotypes across time in unstimulated and LAIV-stimulated organoids. FIG. 2D shows influenza-specific antibodies detected in culture supernatants. FIG. 2E shows the viral neutralizing capacity of antibodies in culture supernatants against two of the four vaccine strains represented in the antigen stimulation (LAIV 2015-2016)-complete cellular and humoral data from n=3 donors (FIGS. 2A-2E) and longitudinal antibody detection data from an additional n=4 donors (FIG. 2D) are shown. FIG. 2F shows influenza M1-specific CD8+ T cell responses in unstimulated and LAIV-stimulated tonsil organoids from four HLA-A2+ donors. Donor ages (in years) are shown in parentheses after donor IDs.

FIGS. 3A-3F show diversity and maturation of the influenza response. FIG. 3A shows confocal microscopy images of a day-4 LAIV-stimulated organoid culture and B cell areas with light zone (LZ) and dark zone (DZ) organization. FIG. 3B shows single-cell RNA-seq of B cells from day-0 tonsil cells and organoid cultures from days 5 and 9 from one donor—the fold change values in genes and antibodies (Ab) highly expressed in GC compared to naive B cells (at least 1.5-fold increase) were plotted from day-0 (d0) B cells and day-5 (d5) LAIV-stimulated B cells. FIG. 3C shows median fluorescence intensity (MFI) for protein-level AID expression (top) and the relative proportions of B cell subsets (bottom) on days 0, 4 and 7 of organoid culture—Box plots show median values with hinges representing the first and third quartiles and whiskers representing the highest and lowest value that is within 1.5 times the interquartile range of the hinges (n=4 donors). FMO, fluorescence minus one. FIG. 3D shows single-cell BCR sequencing of high-affinity A/California 2009 H1N1 HA-specific plasmablasts and GC B cells from one representative donor. High-affinity B cells specific for HA from day-0 (black points) and day-7 LAIV-stimulated organoid cultures (green, blue and red points representing IgM, IgA and IgG isotypes, respectively) are shown—clonal families are represented by open shading. FIG. 3E shows organoid cultures pre-depleted of high-affinity A/California HA+ B cells and non-naive B cells produce new HA+ B cells—effective depletion on day 0 of HA+ B cells and non-naive populations was confirmed by post-sort analysis, and new HA+ B cells were detectable on day 10—data shown are representative plots from one of n=4 donors. FIG. 3F shows influenza-specific antibodies were detected in depleted organoid cultures from three of n=4 donors tested. Donor ages (in years) are shown in parentheses after donor IDs.

FIGS. 4A-4E show somatic hypermutation and antigen-directed affinity maturation supported in tonsil organoids. FIG. 4A shows BCR repertoire analysis workflow on cultures prepared from tonsil cells depleted of high-affinity HA+ and total non-naive B cells (n=6 donors). FIG. 4B shows Influenza vaccine-specific and A/California 2009 H1N1 HA-specific antibodies after 7 d in unstimulated and LAIV-stimulated cultures. FIG. 4C shows Somatic hypermutation as measured by the number of nucleotide mutations from the germline heavy-chain BCR sequences—data are mean±s.e.m. and significance was calculated with two-sided Welch t-tests. FIG. 4D shows clone size (as measured by the number of RNA molecules per lineage) and diversity (as measured by the number of unique RNA molecules per lineage) in A/California 2009 H1N1 HA+ B cell lineages compared to non-A/California HA+ lineages were measured in n=6 donors—specificity for A/California HA was inferred from the pool of known day-0 HA+ BCRs. Box plots show median values with hinges representing the first and third quartiles and whiskers representing the highest and lowest value that is within 1.5 times the interquartile range of the hinges. Each overlaid point represents an individual lineage. Significance values shown were calculated using two-sided Welch t-tests. Donor ages (in years) are shown in parentheses after donor IDs. FIG. 4E shows development of HA specificity in a BCR lineage from one donor. The size of the node represents the number of RNA molecules detected, the distance between nodes is proportional to the edit distance (nucleotide) between them, and the color reflects edit distance (amino acid) to the nearest known A/California HA-specific sequence, with light blue representing an exact match. The root sequence, major node and a subset of the major node with IgG class switch are highlighted.

FIGS. 5A-5H show depletion studies that reveal cell types required for plasmablast differentiation, specific antibody secretion, and antibody affinity in organoid cultures. FIGS. 5A-5B show the effect of cell subset depletion on (FIG. 5A) plasmablast differentiation and (FIG. 5B) specific antibody secretion. Fold change in plasmablast frequency is relative to sorted cells reconstituted with the depleted cell type. Sort depletion experiments were performed on cells from n=6 (naive B cells and non-naive B cells), n=2 (total B cells and plasmacytoid dendritic cells or pDCs) or n=3 (myeloid cells+pDCs, regulatory T (Treg) cells, memory B cells and plasmablasts) donors. FIGS. 5C-5D show the effect of CD4+ depletion on (FIG. 5C) plasmablast differentiation and (FIG. 5D) antibody production after 7 d in organoid culture (n=15 donors). Antibodies against influenza were not detected in unstimulated controls. Paired, two-sided Wilcoxon signed-rank tests were used to determine the significance values. FIG. 5E shows dependence on CD4+ cells for influenza-specific antibody secretion was associated with age. Intact or CD4+ depleted, LAIV-stimulated cultures were stratified into ages 5 years and under (red) or older than 5 (cyan). Box plots show the median values with hinges representing the first and third quartiles and whiskers representing the highest and lowest value that is within 1.5 times the interquartile range of the hinges. Unpaired, two-sided Mann-Whitney U tests were used to calculate significance. FIG. 5F shows biolayer interferometry binding data from intact or CD4+-depleted cultures. Dissociation rates as measured by kd (1/s) are shown for four donors and detailed binding traces from one representative donor. Data were collected from antibodies binding full-length A/California 2009 H1N1 HA or head domain only. Stem-specific antibodies were detected in two donors but only under CD4-containing conditions. Colors are matched to donor data shown in c and d. FIG. 5G shows minimal cell requirements to support an influenza B cell response. Individual cell types were sorted and then recombined as shown to test for plasmablast differentiation and antibody secretion after 7 d of stimulation with LAIV (n=7 donors). FIG. 5H shows contribution of naive versus memory CD4+ T cells in B cell differentiation and influenza-specific antibody secretion under minimal cellular conditions. Naive B cells, APCs and CD45− cells were sorted and combined with equal numbers of either naive (CD45RA+CD27+) or memory (all non-naive) CD4+ T cells for 10 d in the presence of LAIV (n=5 donors).

FIGS. 6A-6G show the ability to respond to non-influenza antigens and immunomodulation with adjuvants. FIGS. 6A-6B, show plasmablast differentiation in response to MMR vaccine stimulation in tonsil organoid cultures (FIG. 6A) and their corresponding MMR-specific IgG production (FIG. 6B). Organoid cultures from seven donors were harvested on day 11 for flow cytometry analysis and antibody secretion. Significance values were calculated using paired, two-sided Wilcoxon signed-rank tests. Donor ages are shown in parentheses. FIG. 6C shows PE-specific B cells were detected at low levels in unstimulated cultures (0.05% of total B cells) and were increased with PE stimulation but not with an irrelevant antigen (n=3 donors; day 11 after stimulation). FIG. 6D shows PE-specific B cell frequencies were enhanced by stimulation in the presence of alum (n=4 donors; day 10 after stimulation). Representative flow cytometry plots are shown from one donor. Individual points are shown for each donor. Treatment groups were tested with paired, two-sided t-tests to determine significance values. FIG. 6E shows tonsil organoid responses to rabies vaccine, as indicated by pre-GC, GC and plasmablast B cell phenotypes and antigen-specific IgM after 14 d of culture. NP, nucleoprotein. P values between treatment groups were determined with paired, two-sided t-tests (n=10 donors). FIG. 6F shows lung-draining lymph nodes and spleen organoids were stimulated for 14 d with rabies vaccine. FIG. 6G shows tonsil organoids were stimulated with vaccine candidates for SARS-CoV-2 (n=12 donors). Ad5 vectors containing either spike protein (S), spike and nucleocapsid protein (SN) or the S1 spike subunit with nucleocapsid (S1N) were used for stimulation, and responses were measured after 14 d in culture. Box plots show the median values with hinges representing the first and third quartiles and whiskers representing the highest and lowest value that is within 1.5 times the interquartile range of the hinges. Culture supernatants were collected and tested for antigen-specific IgM, IgG and IgA. One donor that was positive for multiple proteins is shown. An irrelevant adenoviral vector with norovirus VP1 was used as a control. Paired, two-sided Wilcoxon signed-rank tests were used to calculate P values shown (not adjusted for multiple comparisons).

FIGS. 7A-7D show characteristics of B cells from tonsil organoid cultures. FIG. 7A shows Flow cytometry gating scheme on representative unstimulated and LAIV-stimulated cultures. FIG. 7B shows representative ELISpot wells for detection of Ab-secreting cells with influenza vaccine specificity. The number of spots detected are shown in the corner of each well. FIG. 7C shows a comparison of organoid cultures grown in standard flat-bottom wells vs. transwells. Data shown are from day 7 LAIV-stimulated cultures (n=6 donors). p values shown were determined with paired Wilcoxon signed-rank two-sided tests. Boxplots show median values with hinges representing first and third quartiles and whiskers representing the highest and lowest value that is within 1.5× the interquartile range of the hinges. FIG. 7D shows a correlation between specific Ab secretion and plasmablast frequency in influenza-stimulated and unstimulated cultures on day 7. Detection of influenza-specific IgG antibodies by semi-quantitative indirect ELISA. Optical density was corrected for background using unspent culture medium as a control. Each donor (n=15) was tested under four conditions: no stimulation, IIV-stimulated, LAIV-stimulated, or H1N1 WT virus-stimulated. Plasmablast (CD19+ CD38+++ CD27+) frequencies were determined by flow cytometry.

FIGS. 8A-8E show confocal imaging showing distribution of different cell types and their interactions in tonsil tissue and organoid cultures. Merge does not include DAPI staining for clarity. FIG. 8A shows one fresh tonsil tissue stained with panels of markers to define 1 and B cell areas and GC structure. FIG. 8B shows day 4 unstimulated organoid stained for 1 and B cell markers. FIG. 8C shows day 4 LAIV organoid stained for germinal center markers. FIG. 8D shows day 4 unstimulated organoid stained for B and 1 cell distribution. FIG. 8E shows day 4 LAIV organoid stained for markers of 1 follicular helper cells. For organoid cultures (as shown in FIGS. 8B-8E), representative examples are shown from organoid cultures derived from three different donors.

FIGS. 9A-9B show the quantification of organoid organization and function. FIG. 9A shows the quantification of CXCR4 and CD83 expression levels (mean intensity, left panel) and percent positive (percentage of CD20+B cells, right panel) in day 4 tonsil organoids from one donor. Two areas of an LAIV-stimulated organoid (top GC and bottom GC as also shown in FIG. 3A) and a representative GC-organized area from an unstimulated organoid were used for the calculation. FIG. 9B shows representative intracellular AID flow cytometry staining profiles. Data shown are from a day 4, LAIV-stimulated organoid culture. Total B cells were subsetted based on CD38 and CD27 expression and are shown as individual profiles in red compared to a ‘no AID primary antibody’ FMO control in grey.

FIGS. 10A-10B show B cell receptor sequencing from HA-specific B cells. FIG. 10A shows phylogenetic trees from two donors are shown (top). Day 0 clones are indicated by black points. Different isotypes (IgM, IgA, IgG) are indicated by color (green, blue, and red respectively). Oligoclonal populations and clonal families from single cell data are represented by larger points (open circles). Clonal families were defined as BCR sequences that use the same V and J genes and have at least 70% amino acid identity in the CDR3 region for heavy and light chains. FIG. 10B shows FACS staining and B cell phenotypes of HA-specific B cells compared to the total B cell pool.

FIG. 11 shows tracking of individual heavy-chain BCR lineages before and after LAIV stimulation in tonsil organoids show evidence of isotype switching. Immunoglobulin heavy-chain gene rearrangements for each isotype were sequenced from total memory B cells, germinal center B cells, and plasmablasts sorted from cultures of four donors on days 0 and 7. Heavy-chain BCR sequences from lineages that contained members as only IgM on day 0 and as only isotype-switched on day 7 were compared for their somatic hypermutation levels. For each lineage, the mean SHM was calculated for day 0 IgM members and for day 7 switched members, and the difference between these means was plotted. Lineages with increased mutation are shown in gray and those with decreased mutation in black.

FIG. 12 shows depletion of pre-existing HA-specific and non-naive B cells do not prevent production of new high-affinity HA+ B cells after organoid culture. B cells with high affinity BCRs for A/California 2009 HA+ and all non-naive B cells were depleted by FACS and depletion was confirmed by post-sort analysis. After 10 days in organoid culture, cells were harvested, re-stained for A/California HA+ B cells, and run on a flow cytometer. n=4 donors were tested.

FIGS. 13A-13C show effects of cell depletion on influenza-specific antibodies and their affinities. FIG. 13A shows that rescue of plasmablast and Ab responses to LAIV stimulation by supplementing pDC-depleted cultures with type I IFNs. FIG. 13B shows the frequency of CD4+ T cells (of live cells) in intact and CD4-depleted cultures on day 7. CD4+ cells were depleted by positive selection with magnetic particles. FIG. 13C shows a biolayer interferometry data indicating Ab affinity for A/California 2009 H1N1 HA full length, head-specific, and stem domains.

FIG. 14 shows SARS-CoV2-specific Abs detected in organoid cultures stimulated with Ad5 vectored vaccine candidates. Day 14 post-stimulation culture supernatants were tested for the presence of SARS-CoV2-specific IgM, IgG, and IgA in n=12 donors. Abs were detected using a protein microarray. Individuals donors are represented by symbols. The signal intensities shown were background subtracted based on unstimulated control cultures from the same donor. Boxplots show median values with hinges representing first and third quartiles and whiskers representing the highest and lowest value that is within 1.5× the interquartile range of the hinges.

FIG. 15 shows an example of a computer system configured to implement methods provided herein.

### DETAILED DESCRIPTION OF THE INVENTION

Disclosed herein are methods, systems, and devices capable of recapitulating an immune responses and function. In some embodiments, the methods, systems and devices can comprise immune cell structures capable of, for example, modeling adaptive immune responses including antigen-specific hypermutation, affinity maturation and class switching of B cells. In some embodiments, the immune cell structures can comprise an in-vitro cell cluster comprising lymphoid cells. In some embodiments, the lymphoid cells can be derived from tonsil tissue. In some embodiments, the in-vitro cell cluster can be in contact with a cell culture media in a Transwell system, with the cells suspend in media on a porous membrane. In some embodiments, the in-vitro cell culture can be held at a temperature at or around ninety-eight degrees fahrenheit. In some embodiments, the in-vitro cell cluster can be exposed to one or more adjuvants. In some embodiments, the in-vitro cell cluster can be exposed to one or more antigens. In some embodiments, the antigen can be a protein, carbohydrate, glycoprotein or fragment thereof. In some embodiments, the antigen can be a viral protein, a growth factor, a cancer related protein, or an auto-immune disease related protein.

In some embodiments, the in-vitro cell cluster can comprise a spatial organization of lymphoid tissue. In some embodiments, the spatial organization can comprise a germinal center. In some embodiments, the spatial organization can comprise an aggregate of T-cells and a distribution of rare but essential non-lymphoid cells. In some embodiments, the in-vitro cell cluster can be configured to maintain a spatial organization and cellular respiration for at least 24 hours.

In some embodiments, the in-vitro cell cluster can be configured to perform one or more of: hypermutation maturation, affinity maturation, plasmablast differentiation, class switching recombination, and antigen-specific antibody production. In some embodiments, the cells can be configured to differentiate to form said in-vitro cell cluster upon exposure to an antigen. In some embodiments, the germinal center can comprise antigen presenting cells (APCs) and T-cells at least partially surrounding said functional germinal center. In some embodiments, the APCs can comprise B-cells or dendritic cells. In some embodiments, the dendritic cells can comprise follicular dendritic cells. In some embodiments, the B-cells can comprise CD38+ B-cells. In some embodiments, the B-cells can comprise CD27+ B-cells. In some embodiments, the T-cells can comprise αβ type T-cells. In some embodiments, the T-cells can comprise CD8+ T-cells. In some embodiments, the T-cells can comprise CD4+ T-cells. In some embodiments, the T-cells can comprise γδ T cells

### Definitions

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. A human may be an infant, a toddler, a child, a young adult, an adult or a geriatric. A human can be more than about 1, 2, 5, 10, 20, 30, 40, 50, 60, 65, 70, 75, or about 80 years of age.

Cell Culture Conditions

In some embodiments, the in-vitro cell cluster can be in contact with a media. In some embodiments, the media can comprise one or more components. In some embodiments, the first component can comprise a media component comprised of AIM V, IMDM, MEM, DMEM, RPMI 1640, Alpha Medium or McCoy's Medium, or an equivalent known culture medium component. In some embodiments, the second can be a serum component which can comprise human serum, fetal bovine serum, or horse serum. In some embodiments, the third component can be an activating factor which can comprise B-cell activating factor (BAFF). In some embodiments, the fourth component can be antibiotic to prevent microbial growth. In some embodiments, the fifth component can be basal media supplements which can comprise nonessential amino acids, sodium pyruvate, insulin/selenium/transferrin cocktail, growth factors, hormones, or cytokines. In some embodiments, the media can be supplemented with fetal calf serum. In some embodiments, the media can be replaced every one day, two days, three days, or four days.

Antigens

In some embodiments, the cells can be configured to differentiate to form said in-vitro cell cluster upon exposure to an antigen. In some embodiments, the antigen can be any substance that binds to an antibody. Antigen can be originated from the environment or formed inside the body. In some embodiments, the antigen can be a peptide, protein or fragment thereof, polysaccharides, lipids, nucleic acids, or other biomolecules. In some embodiments, the protein can be a viral protein, a bacterial protein, a growth factor, a cancer related protein, a cancer related peptide, an auto-immune disease related protein, an auto-immune disease related peptide, or fragment thereof.

In some embodiments, the antigen can be a protein or fragment thereof. In some embodiments, the protein can be a viral protein, a growth factor, a cancer related protein, a bacterial antigen, a fungal antigen, or an auto-immune disease related protein. In some embodiments, the viral protein can be derived from a virus. In some embodiments, the viral protein can be derived from, for example, a helical virus, a polyhedral virus, a spherical virus, or a complex virus. In some embodiments, the antigen can be a virus. In some embodiments, the virus can be, for example, a coronavirus or a flu virus.

Adjuvants

Immune responses of the immunological organoid model as described herein can be enhanced by the use of an adjuvant or derivative thereof. In some embodiments, the adjuvants can comprise, for example, aluminum salts, Freund's adjuvant, Poly-IC, Poly-ICLC, MDP, MPL, CpG ODN, Virosome, MF59, AS01, Flagellin, R837/R848, AS04, AS02, AS03, mineral adjuvants such as aluminum hydroxide, phosphate adjuvants, calcium phosphate adjuvants, imiquimod, ISA51, or any combination thereof.

In some embodiments, the lymphoid organoid can be incubated with an antigen and an adjuvant. In some embodiments, the lymphoid organoid can be incubated with an antigen before introducing the adjuvant. In some embodiments, the lymphoid organoid can be incubated with an antigen and/or adjuvant for at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, or more. In some embodiments, the incubation can be for at least 48 hours. In some embodiments, the incubation with one or more antigens can increase the percent of antigen specific B-cells by at least 1%, 2%, 3%, 4%, 5%, 6%, 7%, 8%, 9%, 10%, 11%, 12%, 13%, 14%, 15%, 20%, 25%, 30%, 35%, 40%, 50%, 60%, 65%, 70%, 75%, 80%, 85%, 90%, 95%, 100% or more as compared to before incubation.

In some embodiments, the incubation with one or more antigens can increase a number of nucleotide mutations from a germline heavy chain BCR sequence by at least 1%, 2%, 3%, 4%, 5%, 6%, 7%, 8%, 9%, 10%, 11%, 12%, 13%, 14%, 15%, 20%, 25%, 30%, 35%, 40%, 50%, 60%, 65%, 70%, 75%, 80%, 85%, 90%, 95%, 100% or more as compared to before incubation.

In some embodiments, the incubation with one or more antigens can increase a percent of antibody secreting B-cells amongst the total population of B cells by at least 1%, 2%, 3%, 4%, 5%, 6%, 7%, 8%, 9%, 10%, 11%, 12%, 13%, 14%, 15%, 20%, 25%, 30%, 35%, 40%, 50%, 60%, 65%, 70%, 75%, 80%, 85%, 90%, 95%, 100% or more as compared to before incubation.

Cell Types

In some embodiments, the immunological organoid model can comprise an in-vitro cell cluster that includes, but is not limited to, germinal centers. In some embodiments, the in-vivo cell cluster can comprise other cell clusters, for example, the distinct T cell zone or non lymphoid cells. In some embodiments, the organoids described herein can be derived from tonsil tissue. In some embodiments, the organoid can comprise a germinal center. In some embodiments, the organoid can comprise a T-cell aggregate. In some embodiments, the method can further comprise obtaining said lymphoid cells from a subject. In some embodiments, the lymphoid cells can be obtained by biopsy, swab, or aspiration.

Germinal Center Features

In some embodiments, the in-vitro cell cluster can be configured to perform one or more of: somatic hypermutation, affinity maturation, plasmablast differentiation, class switching recombination, and antigen-specific antibody production. In order to develop antigen specificity, in the germinal center, B cells can undergo a process called somatic hypermutation where point mutations are introduced into B cell receptor (BCR) gene sequence of the antibody variable regions of both the heavy and light chains at a very high rate compared to the background mutation rates observed in other genes. These mutated B cells are different from each other in specificity of antigen. To ensure high affinity of antibody production, mutated B cell are further selected based on the binding affinity of their receptors to antigen from follicular dendritic cells (FDCs), macrophages, or dendritic cells. Those B cells that have a negative effect on antigen binding undergo apoptosis while those with positive binding affinity are selected. This process is called affinity maturation. These two events result in a generation of B cells whose BCRs bind to specific antigen with high affinity. Selected B cells then differentiate into memory B cells or plasmablasts, which are also known as antibody secreting cells (ASC). Plasmablasts produce a large amount of antibodies during the first wave before undergoing apoptosis within a few days while memory B cells provide longer immunity. Memory B cells can also secrete different class of antibodies or immunoglobins (Ig) via a process called class switching recombination. During this process, a DNA recombination process of the constant region of the antibody heavy chain is changed while the variable region of the heavy chains remains the same. As a result, the antibody retains affinity for the same antigen but can interact with different effector molecules.

In some embodiments, the germinal center can comprise antigen presenting cells (APCs). In some embodiments, the germinal center can be at least partially surrounded by T-cells. In some embodiments, the APCs can comprise B-cells, macrophages, or dendritic cells. In some embodiments, the dendritic cells can comprise follicular dendritic cells, pDC. The B-cells can comprise, for example, CD3− B-cells, CD45+ B-cells, CD19+ B-cells, CD38+ B-cells, CD38− B-cells, or CD27+ B-cells. In some embodiments, the B-cells can comprise transitional B-cells, naïve B-cells, plasma B-cells, memory B-cells, pre-GC B cells, GC B cells, plasmablasts. In some embodiments, the T-cells can comprise, for example, helper T-cells, follicular helper T-cells, follicular regulatory T-cells, cytotoxic T-cells, memory T-cells, regulator T-cells, natural killer T-cells, mucosal associated invariant T-cells, gamma delta T-cells. In some embodiments, the T-cells can comprise CD8+ T-cells, CD4+ T-cells.

In some embodiments, the germinal center can comprise a dark zone and a light zone. In some embodiments, the dark zone can contain about 5%, 10%, 15%, 30%, 40%, 50%, 60%, 70%, 80%, 90%, 100%, or more CXCR4+ B cells compared to the light zone. In some embodiments, the light zone can contain about 5%, 10%, 15%, 30%, 40%, 50%, 60%, 70%, 80%, 90%, 100%, or more CD83+ B cells compared to the dark zone. In some embodiments, at least about 1%, 2%, 3%, 4%, 5%, 10%, 15%, 30%, 40%, or more cells in the dark zone can be CXCR4+ B cells. In some embodiments, at least about 1%, 2%, 3%, 4%, 5%, 10%, 15%, 30%, 40%, or more cells in the light zone can be CD83+ B cells. In some embodiments, the germinal center can comprise at least about 1%, 2%, 3%, 4%, 5%, 10%, 15%, 30%, or more CD83+ B-cells. In some embodiments, the germinal center can comprise at least about 1%, 2%, 3%, 4%, 5%, 10%, 15%, 30%, or more CXCR4+ B-cells. In some embodiments, the light zone can contain about 5%, 10%, 15%, 30%, 40%, 50%, 60%, 70%, 80%, 90%, 100%, or more CD86+ cells. CXCL12-expressing reticular cells (CRCs) are stromal cells known to reside in the dark zone. In some embodiments, the dark zone can contain about 5%, 10%, 15%, 30%, 40%, 50%, 60%, 70%, 80%, 90%, 100%, or more CRCs.

Cell Viability

In some embodiments, the spatial organization of lymphoid tissue can be maintained for at least about twelve hours, one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more. In some embodiments, the cell cluster can maintain a cellular respiration for at least about twelve hours, one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more. In some embodiments, cellular respiration can be measured by, for example, a resazurin reduction assay, protease viability marker assay, ATP assay, or luciferase assay.

Uses

In one aspect, disclosed herein is a method for generating antibodies from a lymphoid organoid, comprising placing lymphoid cells in a media to produce said organoid. In some embodiments, the lymphoid organoid can comprise a particular spatial organization of lymphoid tissue. In some embodiments, the lymphoid organoid can comprise one or more germinal centers. In some embodiments, the lymphoid organoid can comprise an aggregate of T-cells. In some embodiments, the method can comprise introducing an antigen to the media to, for example, enhance the immune response of the lymphoid organoid to an antigen. In some embodiments, the method can comprise incubating the lymphoid organoid with an antigen to generate antibodies. In some embodiments, antibodies generated from the lymphoid organoid can be isolated from said lymphoid organoid.

In certain aspects, the methods and systems disclosed herein may be useful for determining a treatment course for a subject. For example, such methods and systems may involve screening a patient for an immune response to a treatment. In some embodiments, the subject can be healthy. In some embodiments, the patient can be positive for a viral infection. In some embodiments, the subject can be positive for an auto-immune disease. In some embodiments, the subject can be positive for a fungal infection. In some embodiments, the subject can be positive for a bacterial infection. In some embodiments, the methods and systems may involve a screen that can be used prophylactically to identify a vaccination for a patient. In some embodiments, the methods and systems may involve a screen that can be used to identify an immune response within a particular population of individuals.

In some embodiments, the methods and systems disclosed herein may be useful for generating monoclonal antibodies for testing and treatment of a variety of diseases, e.g., cancer, autoimmune disease, and/or infectious processes, including viral infection, bacterial infection, microbial infection, or a combination thereof. In some embodiments, the cancer disease can be, for example, chronic lymphocytic leukemia, Hodgkin's lymphoma, Non-Hodgkin's lymphoma, bowel cancer, head cancer, neck cancer, breast cancer, stomach cancer, melanoma, glioblastoma, colorectal cancer, lung cancer, kidney cancer, or ovarian cancer. In some embodiments, the autoimmune disease can be rheumatoid arthritis, Crohn's disease, celiac disease, pernicious anemia, autoimmune vasculitis, myasthenia gravis, Sjogren's syndrome, Graves' disease, Addison's disease, inflammatory bowel disease, systemic lupus erythematosus, Type 1 diabetes, Lupus, Multiple sclerosis, or psoriasis.

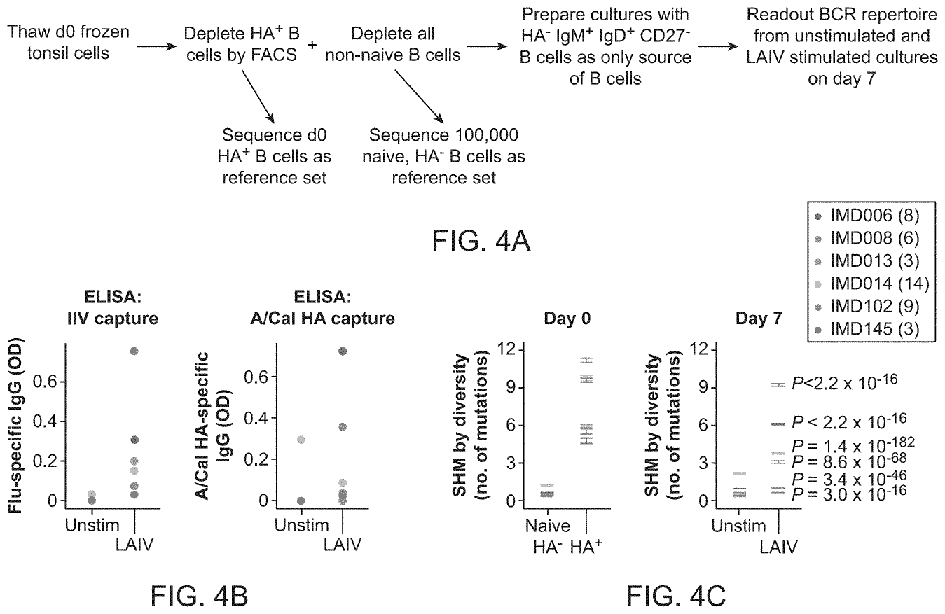

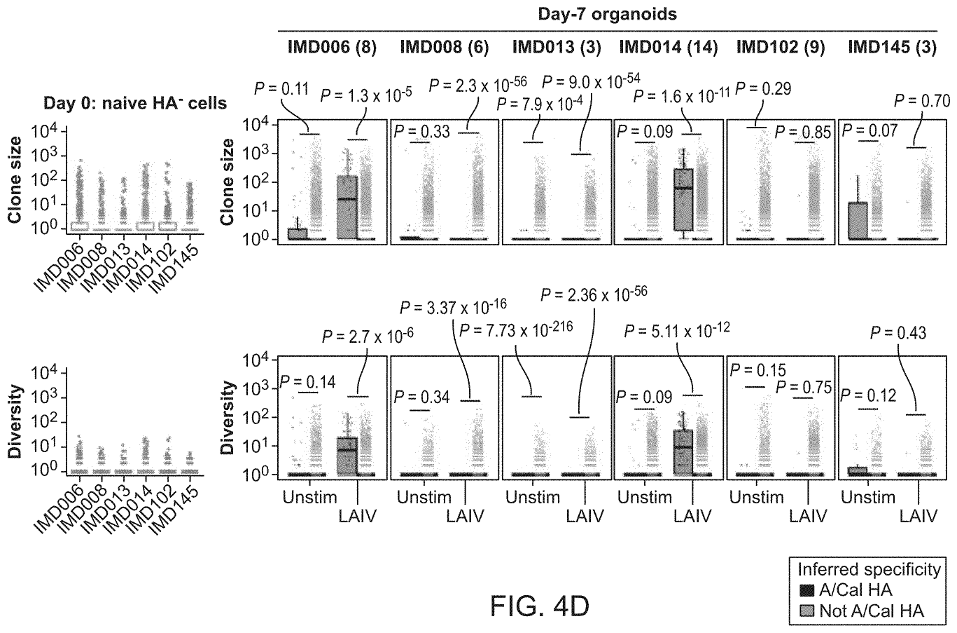

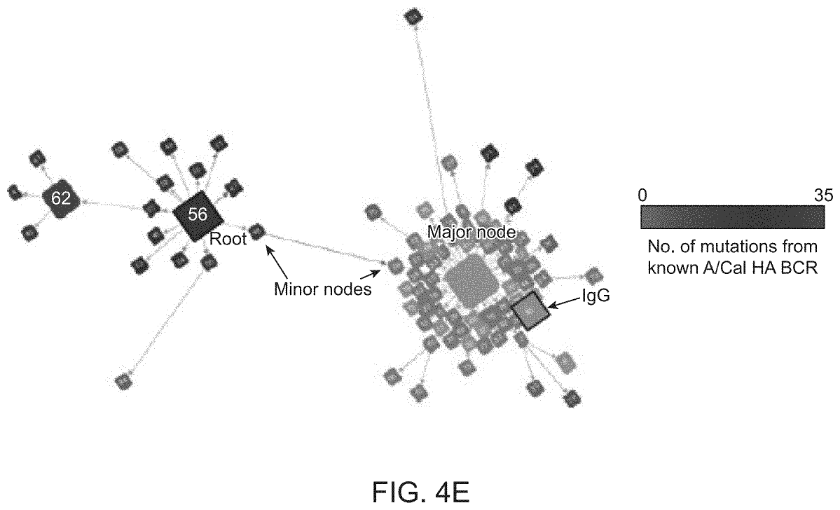

In another aspect, the present disclosure provides an in vitro system supporting one or more of antigen-specific somatic hypermutation, affinity maturation, and class switching of human B cells. Tonsils are a readily available and underutilized source of human lymphoid tissue and comprise the cell types involved in adaptive immunity, for examples, those largely absent from peripheral blood. Tonsil organoids were used to characterize features of the human influenza response and extended findings from previous human and murine studies. Preexisting HA-specific B cells had 5-10 heavy-chain nucleotide mutations (2-4% mutation rate), and LAIV-stimulated organoids prepared with naive-only B cells had 3-9 mutations, which was adequate to make high affinity specific antibodies (FIGS. 4A-4E). These rates are in line with the average 5% mutation rate measured in influenza-vaccinated adults.

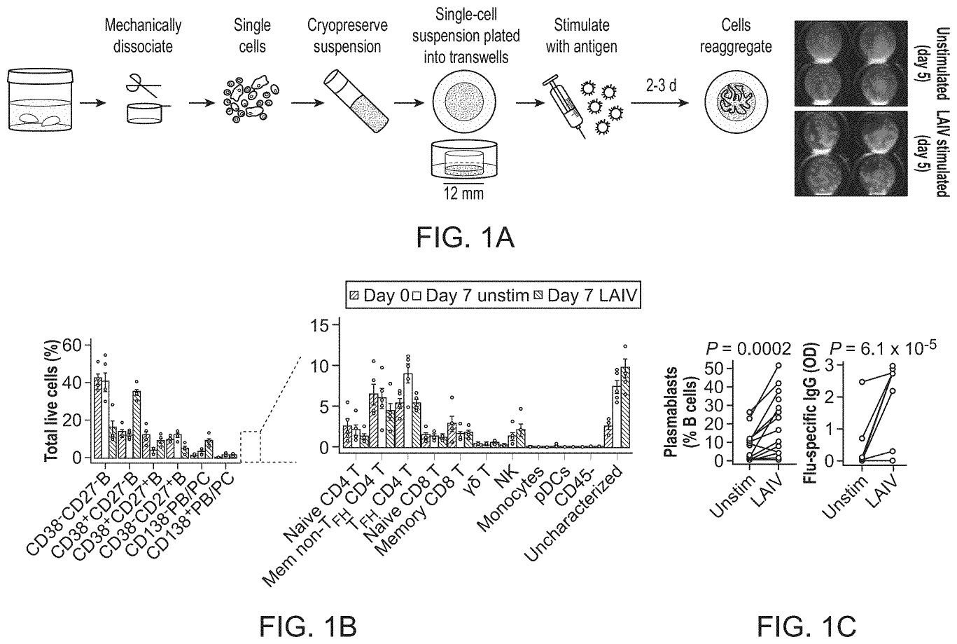

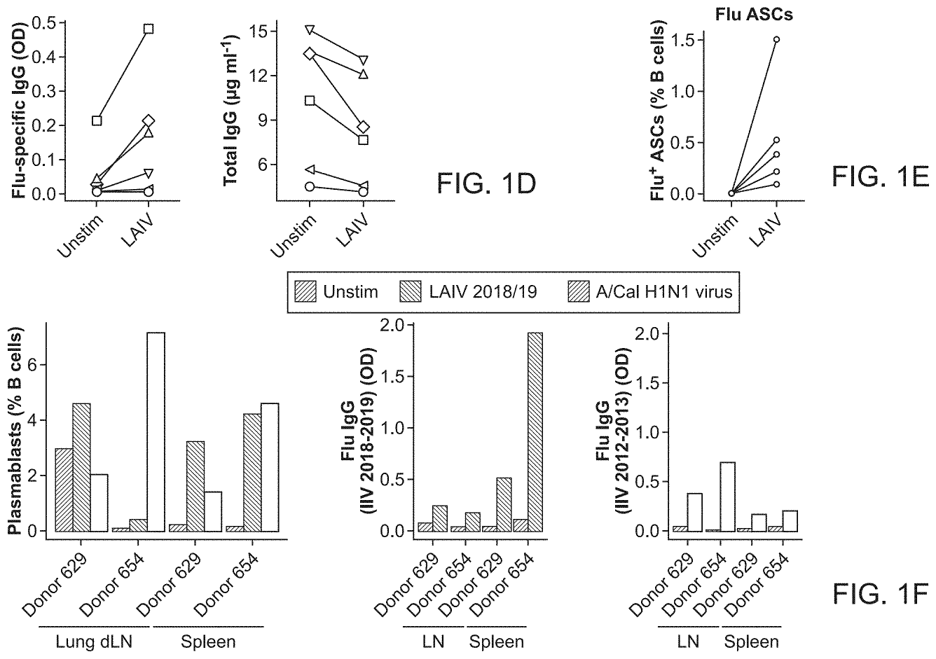

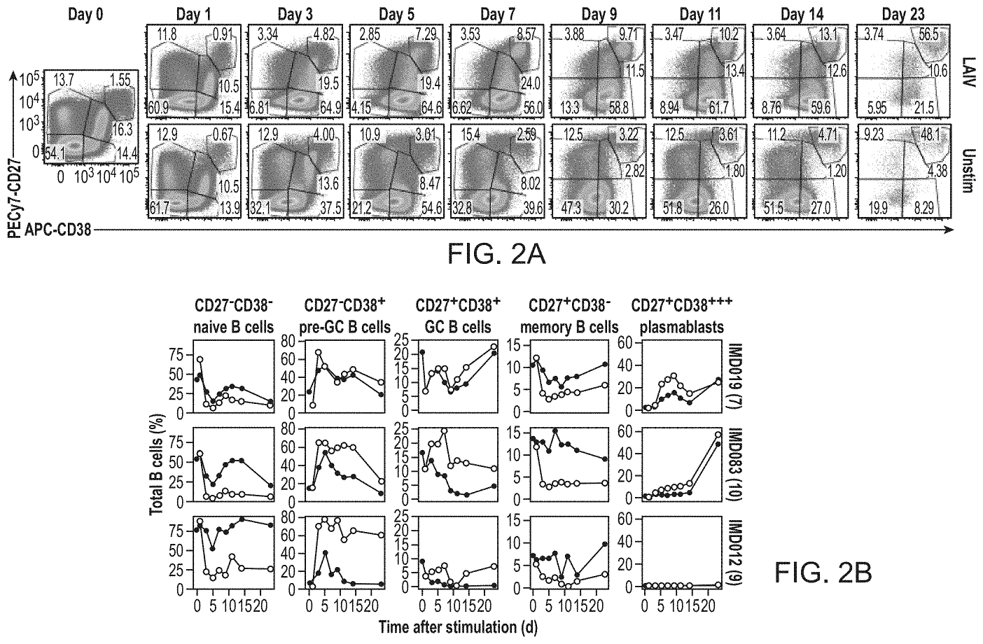

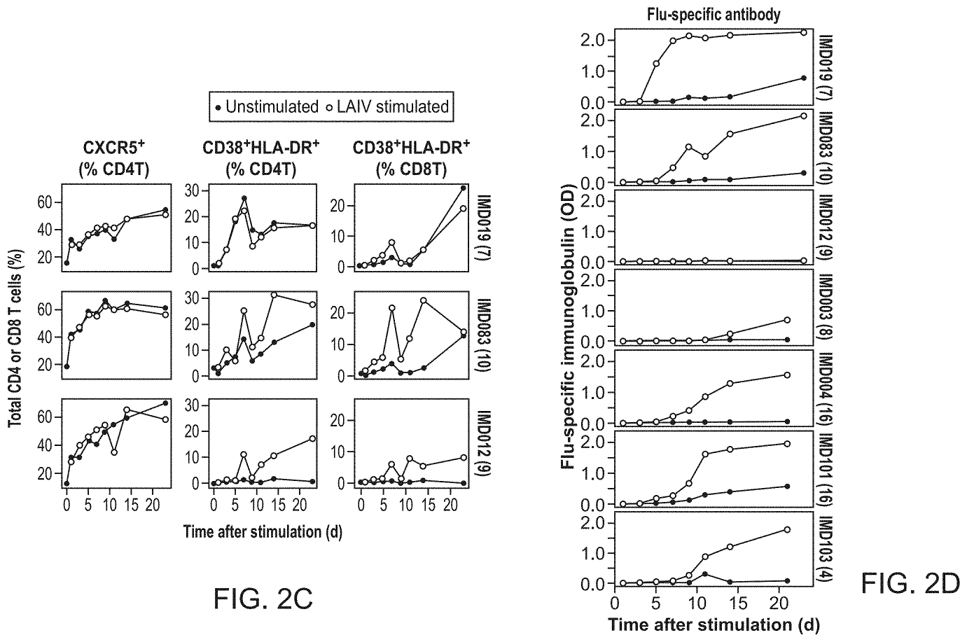

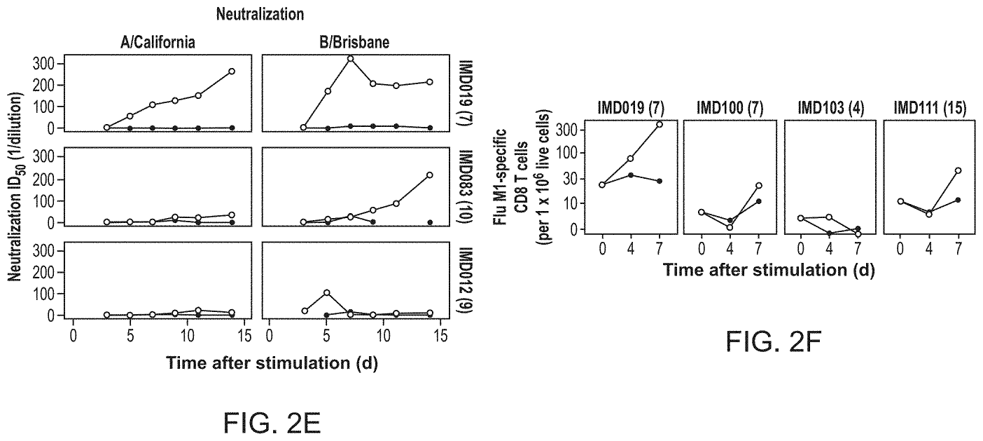

Human immune responses are incredibly variable, as seen in the current SARS-CoV-2 pandemic, and a major goal of vaccine development is to confer protective immunity as broadly as possible. Another useful feature of the organoid system is its ability to assess immune response variability. While the organoids from most donors respond to LAIV, the magnitude and kinetics of those responses vary widely. In a test of 15 donors, organoids prepared from only one donor did not respond to LAIV within 7 d (FIGS. 1A-1F). This individual was young and not vaccinated against influenza, suggesting that they could be naive to influenza, and organoids prepared from their cells might require more time to mount a response. In the longitudinal analyses (FIGS. 2A-2F), a non-responder was identified who was treated with daily inhaled corticosteroids, demonstrating the sensitivity of the system to immunosuppressive agents. Using SARS-CoV-2 vaccine candidates, even more variability was found, with some individuals unable to make B cell and/or T cell responses.

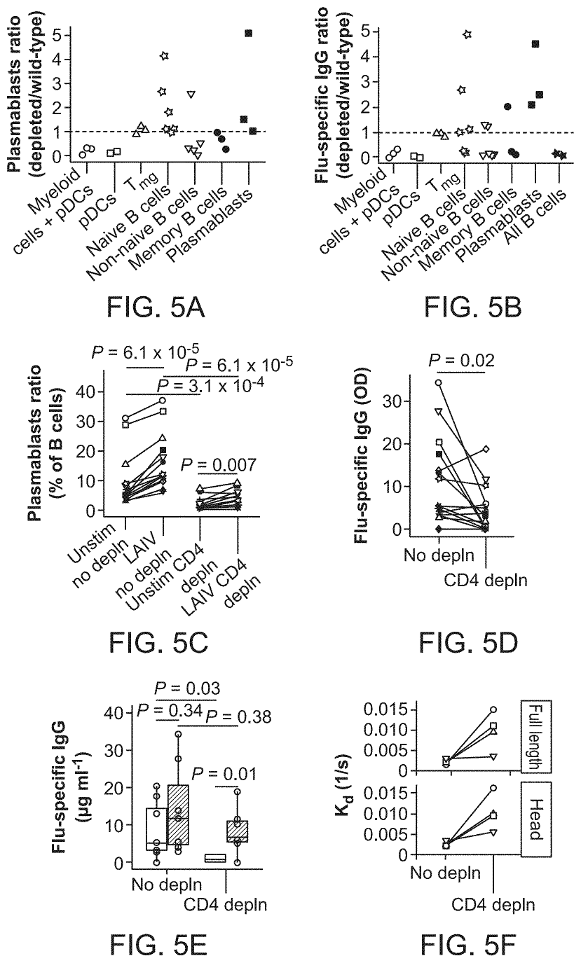

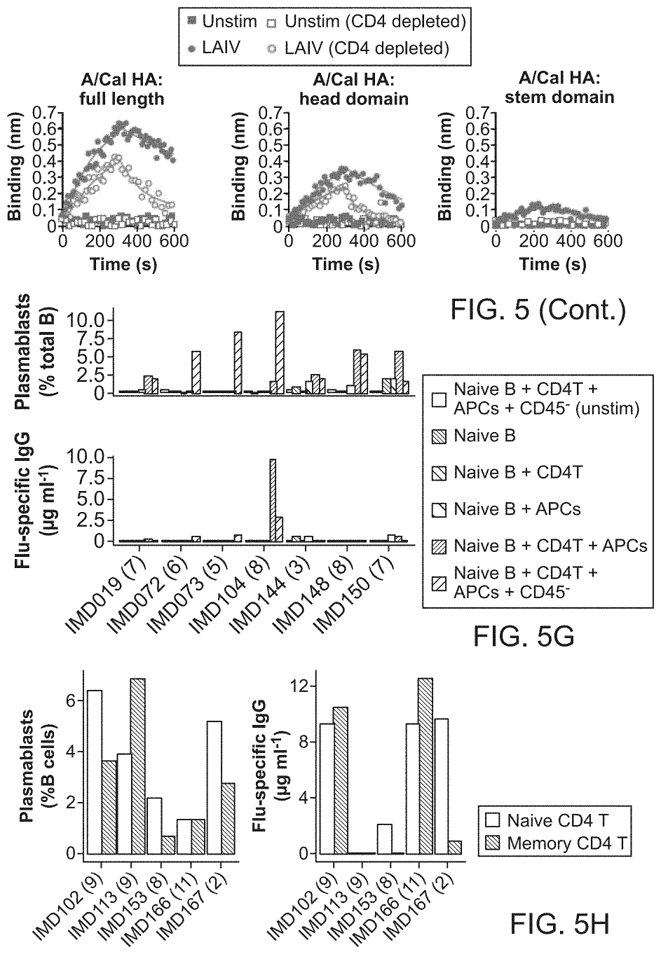

Using depletion studies, the functional relevance of individual immune cell types to the influenza response was established. pDCs were part of the antibody response. The pDCs can be replaced by exogenous type I IFN. These data are consistent with a previous study that found that pDCs induce human blood B cell differentiation through type I IFN41 but contrast with a murine influenza challenge study that showed pDCs were dispensable to the response, highlighting a difference between murine and human systems. A role for preexisting plasmablasts/plasma cells in regulating B cell differentiation (FIGS. 5A-5B) was identified. When plasmablasts were depleted at the initiation of the culture, the influenza-specific antibody production was increased compared to wild-type controls. Secondary lymphoid tissues are competitive niches for plasma cell survival outside of the bone marrow, so depletion of preexisting plasma cells may promote antigen-specific plasmablast survival. It was demonstrated that T cell help plays an age-dependent role in the magnitude of the influenza-specific antibody response and showed that CD4+ T cells play an additional role in affinity maturation and B cell selection (FIGS. 5A-5H). Memory CD4+ T cells from older children better sustained a naive B cell response, suggesting that T cell help is refined by in vivo exposure. These findings are in line with an earlier mouse study showing that memory T cells can contribute to GC responses even though naive B cells typically did not in their system. Stem-specific antibodies were also only detectable when CD4+ T cells were present, suggesting that manipulating T cell help is a reasonable strategy to consider for future vaccine design.

Previous efforts to create artificial human lymphoid tissues depended on specialized bioreactors or complex in vitro differentiation strategies. Blood-derived mononuclear cells combined with monocyte-derived dendritic cells can stimulate IgM and cytokine secretion consistent with an adaptive immune response. It is questionable whether in vitro matured dendritic cells recapitulate the conditions that coordinate T and B cell responses in lymphoid tissues. More importantly, a major limitation of bioreactor systems is that they do not show mature GCs, class switching or affinity maturation, likely because many cells required for GC function are absent from peripheral blood mononuclear cells. Recent work illustrated that key aspects of the immune microenvironment can be captured in organoids in several studies that focused on conferring immune protection upon transplantation into mice or recapitulating features of the GC response. In these studies, designer hydrogel scaffolds for B cells combined with engineered fibroblasts were used to reveal transcriptional feedback loops involved in GC formation but lacked antigen specificity. Most recently, maleimide-functionalized hydrogel was shown to support GC B cell activation and an antigen-specific response in murine B cell organoid cultures. However, these methods do not incorporate autologous APCs nor T cells that are both crucial to refine the adaptive immune response in vivo. Due to the need for bioengineered fibroblast cell lines to provide necessary survival signals, these techniques are challenging to translate to human cells because of the diversity of human leukocyte antigen genes and generation of mixed leukocyte reactions. Previous techniques to produce in vitro models of human adaptive immunity have not been widely adopted because of reliance on specialized equipment or challenging technical protocols, poor throughput, lack of evidence for antigen-specific responses, affinity maturation and/or absence of cells known to be crucial to important features of adaptive immunity. Although humanized mice are increasingly used, they are expensive to procure and still have many limitations in terms of recapitulating human immunity.

It has been established that the LAIV is less effective than the inactivated formulation in adults, presumably due to the presence of preexisting antibodies at mucosal sites in non-naive individuals. Such conditions can be mimicked by introducing autologous serum or spiking in influenza-specific antibodies at the initiation of organoid culture.

Kits

A kit may include, but is not limited to, one or more containers housing one or more of the components provided in this disclosure and instructions for use. Specifically, such kits may include, but is not limited to, one or more compositions described herein, along with instructions describing the intended application and the proper use and/or disposition of these compositions. Kits may comprise the components in appropriate concentrations or quantities for running various experiments.

Machine Learning

In some embodiments, the methods and systems disclosed herein can utilize artificial intelligence/machine learning to generate optimal antibodies with high specificity and affinity. In some embodiments, the artificial intelligence/machine learning can be used to predict an antigen that might be specific to certain type of cancer, autoimmune disease or infection. In some embodiments, the predicted antigen can be used in antibody production from the methods and systems disclosed in this invention.

Computer Systems

In one aspect, the present disclosure provides computer systems for implementing methods provided herein. FIG. 15 shows an example of a computer system 1001. In some embodiments, the computer system 1001 includes, but is not limited to, a central processing unit (CPU, also “processor” and “computer processor” herein) 1005, which can be a single core or multi core processor, or a plurality of processors for parallel processing. In some embodiments, the computer system 1001 also includes, but is not limited to, memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. In some embodiments, the memory 1010, storage unit 1015, interface 1020 and peripheral devices 1025 are in communication with the CPU 05 through a communication bus (solid lines), such as a motherboard. The storage unit 1015 can be a data storage unit (or data repository) for storing data. In some embodiments, the computer system 1001 can be operatively coupled to a computer network (“network”) 1030 with the aid of the communication interface 1020. In some embodiments, the network 1030 can be the Internet, an internet and/or extranet, or an intranet and/or extranet that is in communication with the Internet. In some embodiments, the network 1030 in some cases is a telecommunication and/or data network. In some embodiments, the network 1030 can include, but is not limited to, one or more computer servers, which can enable distributed computing, such as cloud computing. In some embodiments, the network 1030, in some cases with the aid of the computer system 1001, can implement a peer-to-peer network, which may enable devices coupled to the computer system 1001 to behave as a client or a server.

In some embodiments, the CPU 1005 can execute a sequence of machine-readable instructions, which can be embodied in a program or software. In some embodiments, the instructions may be stored in a memory location, such as the memory 1010. In some embodiments, the instructions can be directed to the CPU 1005, which can subsequently program or otherwise configure the CPU 1005 to implement methods of the present disclosure. Examples of operations performed by the CPU 1005 can include, but are not limited to, fetch, decode, execute, and writeback.

In some embodiments, the CPU 1005 can be part of a circuit, such as an integrated circuit. In some embodiments, one or more other components of the system 1001 can be included in the circuit. In some cases, the circuit is an application specific integrated circuit (ASIC).

In some embodiments, the storage unit 1015 can store files, such as drivers, libraries and saved programs. In some embodiments, the storage unit 1015 can store user data, e.g., user preferences and user programs. In some embodiments, the computer system 1001 in some cases can include one or more additional data storage units that are external to the computer system 1001, such as located on a remote server that is in communication with the computer system 1001 through an intranet or the Internet.

In some embodiments, the computer system 1001 can communicate with one or more remote computer systems through the network 1030. For instance, the computer system 1001 can communicate with a remote computer system of a user (e.g., remote cloud server). Examples of remote computer systems include, but are not limited to, personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. In some embodiments, the user can access the computer system 1001 via the network 1030.

In some embodiments, methods as described herein can be implemented by way of machine (e.g., computer processor) executable code stored on an electronic storage location of the computer system 1001, such as, for example, on the memory 1010 or electronic storage unit 1015. In some embodiments, the machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

In some embodiments, the code can be pre-compiled and configured for use with a machine having a processer adapted to execute the code, or can be compiled during runtime. In some embodiments, the code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. In some embodiments, machine-executable code can be stored on an electronic storage unit, such as memory (e.g., read-only memory, random-access memory, flash memory) or a hard disk. In some embodiments, “storage” type media can include, but is not limited to, any or all of the tangible memory of the computers, processors or the like, or associated modules thereof, such as various semiconductor memories, tape drives, disk drives and the like, which may provide non-transitory storage at any time for the software programming. In some embodiments, all or portions of the software may at times be communicated through the Internet or various other telecommunication networks. In some embodiments, such communications, for example, may enable loading of the software from one computer or processor into another, for example, from a management server or host computer into the computer platform of an application server. Thus, another type of media that may bear the software elements includes, but is not limited to, optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. In some embodiments, non-volatile storage media include, for example, optical or magnetic disks, such as any of the storage devices in any computer(s) or the like, such as may be used to implement the databases, for example, shown in the drawings. In some embodiments, volatile storage media include, but is not limited to, dynamic memory, such as main memory of such a computer platform. In some embodiments, tangible transmission media include, but is not limited to, coaxial cables; copper wire and fiber optics, including the wires that comprise a bus within a computer system. In some embodiments, carrier-wave transmission media may take the form of electric or electromagnetic signals, or acoustic or light waves such as those generated during radio frequency (RF) and infrared (IR) data communications. In some embodiments, common forms of computer-readable media therefore include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. In some embodiments, many of these forms of computer readable media may be involved in carrying one or more sequences of one or more instructions to a processor for execution.

In some embodiments, the computer system 1001 can include or be in communication with an electronic display 1035 that comprises a user interface (UI) 1040 for providing, for example, an electronic output of identified gene fusions. Examples of UI's include, without limitation, a graphical user interface (GUI) and web-based user interface.

In some embodiments, the methods and systems of the present disclosure can be implemented by way of one or more algorithms. In some embodiments, an algorithm can be implemented by way of software upon execution by the central processing unit 1005.

### EXAMPLES

### Example 1—Preparation of Immune Organoids from Tonsils and Other Lymphoid Tissues

Human tonsil cultures were developed with dissociated cells that reaggregated in culture. Table 1 below shows the characteristics of the tissue donors.

TABLE 1
Characteristics of tissue donors.

| | | | | | recent |
|---|---|---|---|---|---|
| | | race or | indication for | | influenza |
| Donor | sex | ethnicity | surgery | age | vaccination |
| IMD005 | F | Hispanic | ND* | 3 | ND |
| IMD006 | F | Caucasian | ND | 8 | ND |
| IMD008 | F | Hispanic | ND | 6 | ND |
| IMD009 | F | Hispanic | OSA** | 8 | ND |
| IMD010 | M | Caucasian | OSA, otitis media | 2 | ND |
| IMD012 | M | Hispanic | hypertrophy | 9 | yes |
| IMD013 | F | Hispanic | OSA, otitis media | 3 | ND |
| IMD014 | F | Caucasian | ND | 14 | yes |
| IMD019 | F | Hispanic | OSA | 7 | yes |
| IMD021 | M | Hispanic | tonsillitis | 5 | no |
| IMD022 | M | Hispanic | hypertrophy | 6 | no |
| IMD024 | M | Caucasian | OSA | 3 | ND |
| IMD027 | M | Caucasian | ND | 3 | no |
| IMD029 | M | Filipino | ND | 9 | yes |
| IMD030 | F | Caucasian | ND | 2 | no |
| IMD035 | F | Asian | hypertrophy | 3 | yes |
| IMD049 | M | Caucasian | OSA | 6 | ND |
| IMD058 | F | Caucasian | OSA | 3 | ND |
| IMD068 | M | Caucasian | ND | 5 | ND |
| IMD069 | M | Caucasian | OSA | 3 | ND |
| IMD070 | M | Hispanic | OSA | 6 | ND |
| IMD072 | M | Hispanic | OSA | 6 | ND |
| IMD073 | M | Caucasian | OSA | 5 | ND |
| IMD079 | M | Hispanic | velopharyngeal | 7 | ND |
| | | | insufficiency | | |
| IMD080 | M | Asian | OSA | 5 | ND |
| IMD081 | M | Caucasian | OSA | 7 | ND |
| IMD082 | M | Caucasian | sleep-disordered | 2 | ND |
| | | | breathing | | |
| IMD083 | M | Hispanic | OSA | 10 | yes |
| IMD086 | M | Caucasian | OSA | 8 | no |
| IMD087 | F | Caucasian | OSA | 10 | yes |
| IMD088 | F | Hispanic | OSA | 4 | no |
| IMD092 | F | Caucasian | OSA | 5 | no |
| IMD093 | M | Asian, | OSA | 2 | yes |
| | | Caucasian | | | |
| IMD095 | F | Caucasian | OSA | 4 | yes |
| IMD096 | M | Hispanic | OSA | 8 | yes |
| IMD098 | F | Caucasian | OSA | 5 | no |
| IMD099 | M | African | OSA | 5 | no |
| | | American | | | |
| IMD100 | M | Hispanic | OSA | 7 | yes |
| IMD102 | F | Hispanic | OSA | 9 | ND |
| IMD103 | M | Hispanic | OSA | 4 | yes |
| IMD104 | F | Caucasian | hypertrophy | 8 | yes |
| IMD107 | M | Hispanic | tonsillitis | 9 | yes |
| IMD108 | M | Caucasian | OSA | 4 | yes |
| IMD111 | M | Caucasian | OSA | 15 | yes |
| IMD113 | F | Caucasian | OSA | 9 | yes |
| IMD117 | M | Caucasian | OSA | 7 | no |
| IMD120 | F | Caucasian | OSA | 3 | no |
| IMD144 | F | Caucasian | OSA | 3 | yes |
| IMD145 | M | Hispanic | OSA | 3 | yes |
| IMD148 | M | Other | OSA, hypertrophy | 8 | no |
| IMD150 | F | Hispanic | OSA, hypertrophy | 7 | yes |
| IMD151 | M | Hispanic | OSA | 4 | no |
| IMD153 | F | Hispanic | OSA | 8 | yes |
| IMD163 | M | Asian, | OSA | 9 | yes |
| | | Caucasian | | | |
| IMD166 | F | Hispanic | OSA | 11 | yes |
| IMD167 | M | ND | OSA | 2 | yes |
| IMD170 | M | Caucasian | OSA | 7 | yes |
| VIP012 | F | Caucasian | OSA | 33 | no |
| VIP013 | M | Caucasian | OSA | 72 | ND |
| VIP017 | F | Hispanic | ND | 54 | ND |
| LN/spleen 629 | M | ND | NA*** | 53 | ND |
| LN/spleen 654 | M | ND | NA | 64 | ND |
| LN/spleen 655 | M | ND | NA | 65 | ND |
| LN/spleen 665 | F | ND | NA | 75 | ND |
| LN/spleen 680 | F | ND | NA | 44 | ND |
| LN/spleen 690 | F | ND | NA | 59 | ND |

*ND no data
**OSA obstructive sleep apnea
***NA not applicable

Whole tonsils from 150 consented individuals undergoing surgery for obstructive sleep apnea, hypertrophy or recurrent tonsillitis were collected in accordance with the Stanford University Institutional Review Board (IRB). Ethics approval was granted by the Stanford University IRB (protocols 30837 and 47690). Written informed consent was obtained from adult participants and from the legal guardians of children aged 0-17 years; written informed assent was also obtained from children aged 7 years and older. In this cohort, the participants were children aged 2-17 years (n=57) and adults (n=3) who had surgery for obstructive sleep apnea and/or hypertrophy, and overall, tonsil tissue was typically healthy. Whole tonsils were collected in saline after surgery and then immersed in an antimicrobial bath of Ham's F12 medium (Gibco) containing Normocin (InvivoGen), penicillin and streptomycin for 1 h at 4° C. for decontamination of the tissue. Tonsils were then briefly rinsed with PBS and processed as needed for culturing (see below).

Donor lung lymph nodes and spleens were provided by the Gift of Hope Organ and Tissue Donor Network to the University of Chicago. These tissues were determined to have IRB Exempt Status by the University of Chicago IRB. Only de-identified demographic information was obtained.

For cryopreservation of tonsil cells, tissue was dissected into roughly 5 mm×5 mm×5 mm pieces and manually disrupted into a suspension by processing through a 100-μm strainer with a syringe plunger. Enzymatic dissociation was not necessary and did not improve the response to LAIV from cryopreserved cells. Tissue debris was reduced by Ficoll density gradient separation, although this step was not required for tonsil organoid development. After washing with complete medium (RPMI with glutamax, 10% FBS, 1× nonessential amino acids, 1× sodium pyruvate, 1× penicillin—streptomycin, 1× Normocin (InvivoGen) and 1× insulin/selenium/transferrin cocktail (Gibco)), cells were enumerated and frozen into aliquots in FBS+10% DMSO. Frozen cells were stored at −140° C. until use.

For lung-draining lymph nodes and spleen samples, tissues were collected in saline or Hank's Balanced Salt Solution, diced into small pieces and pressed through nylon mesh (Nitex) to break up the tissue. Cells were isolated with Ficoll density gradient separation, washed, enumerated and frozen in FBS+10% DMSO. Frozen samples were stored in liquid nitrogen until use.

For culture of cryopreserved cells, aliquots were thawed into complete medium, enumerated and resuspended to 6×10⁷ cells per ml for larger cultures or 2×10⁷ cells per ml for smaller cultures. Cells were plated, 100 μl per well, into permeable (0.4-μm pore size) membranes (24-well size PTFE or polycarbonate membranes in standard 12-well plates or 96-well polycarbonate membrane plates with single-well receiver trays; Corning or Millipore), with the lower chamber consisting of complete medium (1 ml for 12-well plates, 200 μl for 96-well plates) supplemented with 1 μg ml-1 of recombinant human B cell-activating factor (BAFF; BioLegend). Adding a small amount of BAFF improved total B cell survival (and thus increased overall cell recovery) but was not a requirement for plasmablast differentiation or antibody secretion.

LAIV (1 μl per well, equivalent of 1.6×10⁴ to 1.6×10⁵ fluorescent focus units per strain; FluMist Quadrivalent, Medimmune), wild-type influenza virus (A/California/07/2009 pandemic strain; a gift from H. Greenberg and X.-S. He), MMR vaccine (5 μl per culture; Merck), R-phycoerythrin (1 μg per culture; Thermo Fisher), rabies vaccine (10 μl per culture; Imovax, Sanofi Pasteur) or Ad5-vectored SARS-CoV-2 vaccine candidate (1×10⁸ infectious units per culture; Vaxart) was then added directly to the cell-containing portion of the culture setup. For adjuvant testing, alum (0.01%; InvivoGen, sold as a 2% stock wet gel with 9-11 mg ml-1 stock aluminum content) or imiquimod (2.5 μg ml-1; InvivoGen) was added directly into the culture immediately after antigen addition. Cultures were incubated at 37° C., 5% CO2 with humidity and supplemented with additional medium to the lower wells as necessary. Hypoxic conditions (5% O2) were tested for B cell differentiation and antibody secretion and were not significantly different from cultures maintained at standard incubator oxygen levels (17-21%).

For the Ad5-vectored vaccine candidates, recombinant adenoviral constructs were produced using the publicly available SARS-CoV-2 DNA sequence (GenBank accession no. MN908947.3). Spike and nucleocapsid protein sequences were synthesized and codon optimized for expression in human cells and cloned into the E1 region as previously described. The same vector backbone has been used previously in clinical trials for oral recombinant adenovirus tablets. The Ad-S adenoviral vector contains a spike protein under the human cytomegalovirus (CMV) promoter. The Ad-SN vector contains spike under the CMV promoter and nucleocapsid under the human beta-actin promoter. The recombinant Ad-S1N vector uses a fusion sequence combining the S1 region of the SARS-CoV-2 spike gene (including the native furin site between S1 and S2) with the full-length SARS-CoV-2 nucleocapsid gene. All vaccine candidates were purified by cesium chloride density centrifugation and provided in a liquid form for cell culture experiments.

For organoid preparation, frozen single-cell suspensions from tonsil tissues were thawed and plated at high density into the wells of permeable membrane plates (e.g., transwells) along with the antigen of interest. After several days in culture, reaggregated regions of clustered cells were visible as can be seen in FIG. 1A. The cell composition of the reaggregated cultures were assessed after 7 days in the presence or absence of antigen and the optimized conditions sustained appropriate tonsil cell composition, as can be seen in FIG. 1B.

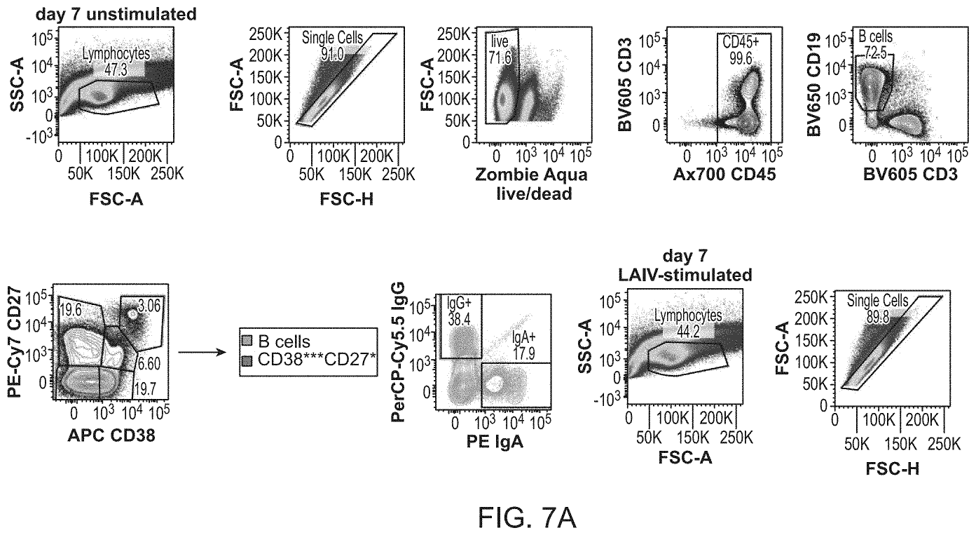

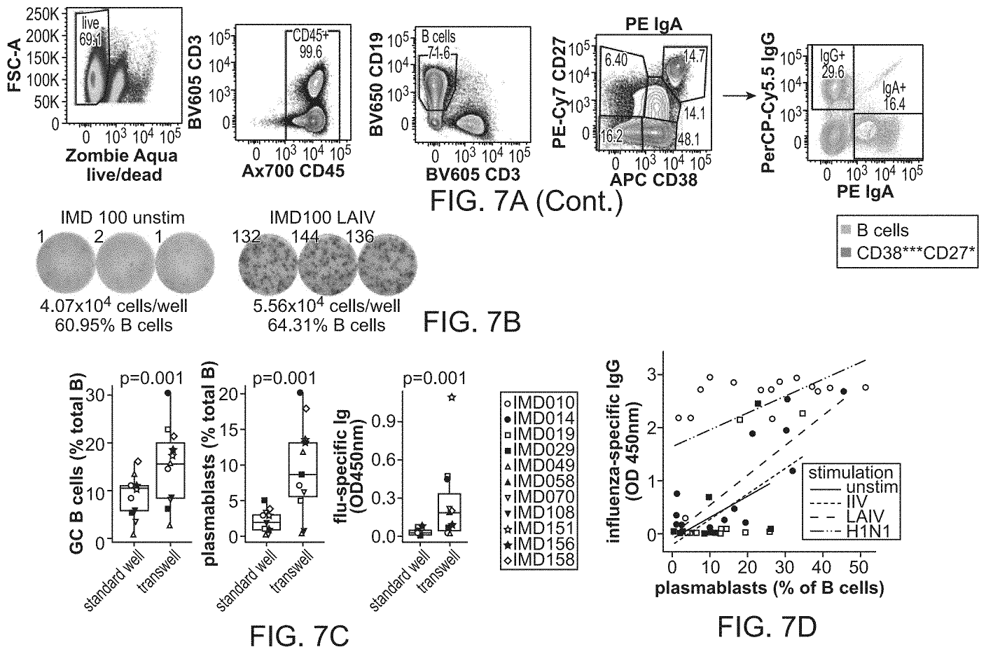

Influenza vaccines and viruses were used as model antigens since much is already known about the features of the human influenza response in vivo. Upon stimulation with live attenuated influenza vaccine (LAIV), there were notable increases in B cell differentiations and a more structures culture morphology developed, suggesting additional activity in response to this immunogen. The capacity of organoid cultures to support B cell maturation and function upon LAIV stimulation was then measured by staining the cultures as can be seen in FIG. 7A.

Flow cytometry. Organoids were harvested from the upper portion of the permeable membranes by rinsing the membranes with PBS. Cells were washed with FACS buffer (PBS+0.1% BSA, 0.05% sodium azide and 2 mM EDTA) and stained at 4° C. with the following anti-human antibodies in the presence of Fc block and live/dead Aqua Zombie stain, all from BioLegend unless otherwise noted: FITC CD138 (1/100), FITC CD116 (1/100), FITC CD21 (1/50), FITC or Ax488 CXCR5 (1/33), PerCP-Cy5.5 CD8 (1/100), PerCP-Cy5.5 CD33 (1/100), PE CD19 (1/100), PE CD56 (1/100), PE gamma-delta TCR (1/100), PE-Cy7 CD27 (1/100), PE-Cy7 CD123 (1/50), PE-Cy7 CD8 (1/100), APC CD38 (1/200), APC HLA-DR (1/100), APC CD27 (1/200), Ax700 CD45 (1/100), Ax700 CD14 (1/100), APC-Cy7 IgD (1/50), APC-Cy7 CD16 (1/100), APC-Cy7 CD45RA (1/100), Pacific Blue HLA-DR (1/100), Pacific Blue PD-1 (1/50), BV605 CD3 (1/100), BV650 CD4 (1/100), BV650 CD19 (1/100), BUV395 IgM (1/20; BD Biosciences) and BUV395 CD45RA (1/20; BD Biosciences).

For AID staining, after surface staining, the cells were fixed and permeabilized (eBioscience) and stained intracellularly with biotinylated anti-AID antibody (1/100; clone mAID-2; eBioscience) followed by PE-streptavidin (eBioscience). A no-AID antibody control was used to discriminate positive signal. All analyzer data were collected on BD LSRII instruments and analyzed using FlowJo (TreeStar).

It was found that after 7 days, plasmablast frequencies were significantly increased compared to unstimulated controls (FIG. 1C; n=15; P=0.0002). Thirteen of the fifteen donors tested, ranging in age from 2-14 years, produced influenza-specific IgG antibodies in only the LAIV-stimulated samples (P<0.0001). Of the two exceptions, one donor had a high response without stimulation and the other was a true nonresponder. The latter donor (a 3-year-old) may have been naive for influenza infection and had no reported influenza vaccination. The production of specific antibody was not a generic effect of stimulation, as total (that is, antigen agnostic) IgG was reduced by LAIV (FIG. 1D). Enzyme-linked immune absorbent spot (ELISpot) analysis was performed on day-7 cultures from five donors and influenza-specific antibody-secreting cells (ASCs) ranging from 0.1-1.5% of total B cells was found (FIG. 1E; representative ELISpot data in FIG. 7B). Almost no influenza-specific ASCs were identified in unstimulated tonsil cultures (<0.02% of total B cells in all donors). The Transwell organoid culture strategy was superior to flat-bottom tissue culture plates of similar surface area for growing tonsil mononuclear cells (FIG. 7C).

Antibody detection by ELISA. For detection of influenza-specific antibodies, ELISA plates (Costar) were coated with 0.1 μg per well of season-matched Fluzone Quadrivalent inactivated influenza vaccine (based on reported total HA content from the manufacturer; Sanofi) to act as the capture antigen. For A/California HA antibody detection, recombinantly expressed soluble HA trimers were used in place of the inactivated vaccine as the capture antigen. Diluted (1:20 or 1:50) culture supernatants were added to coated, blocked plates. A human pan-influenza monoclonal IgG antibody (H1N13-M; Alpha Diagnostics) was used as a standard to estimate specific antibody concentration for experiments where antibody concentration is quantitatively defined. Horseradish peroxidase-conjugated anti-human secondary antibodies to either IgM/IgG/IgA (Sigma) or Fc-IgG alone (Bethyl; adsorbed for other isotypes) were used to detect bound antibodies. Plates were developed with TMB substrate solution (Thermo Scientific), quenched with sulfuric acid and read at 450 nm. Neutralization experiments were performed by Monogram Biosciences with a pseudovirus neutralization assay with HA matching the vaccine antigen strains.

For detection of total IgG, culture supernatants were diluted at 1:500 and assayed by ELISA (Thermo Scientific) following manufacturer's instructions. For detection of MMR-specific IgG, culture supernatants were tested by ELISA (Abcam) following the manufacturer's instructions. Supernatants were diluted at 1:2.5 with the provided sample diluent and serial twofold dilutions out to 1:40 confirmed signal specificity (data not shown). To measure rabies nucleoprotein-specific IgM and IgG, supernatants were diluted at 1:5 with specific IgM and IgG detection kits (Alpha Diagnostic).

Antibody detection by protein microarray. Organoid cultures stimulated with recombinant adenoviral vectors with SARS-CoV-2 sequences were tested for specific antibody production using a protein microarray. The technology has been previously described for the detection of influenza-specific antibodies and was recently adapted to include commercially available SARS-CoV-2 proteins. The SARS-CoV-2 proteins are commercially available from Sino Biological. Briefly, day-14 culture supernatants were diluted 1:1 with blocking buffer and incubated for 30 min. Then, diluted samples were added to the microarray for overnight hybridization. The array was washed three times with Tris-buffered saline containing Tween20, then treated with Qdot-conjugated anti-IgM, IgG or IgA secondary antibodies for 2 h. After another three washes, the array was dried and read, with signal intensities representing relative antibody quantities bound to each protein spot.

Also tested was whether the organoid culture strategy could support human lung-draining lymph node and spleen samples (FIG. 1F). These organoids responded to both LAIV and wild-type A/California H1N1 influenza virus by producing influenza-specific antibodies at levels substantially above those of control cultures and equivalent to tonsil organoids.

### Example 2: Longitudinal Analysis of Tonsil Cultures in Response to LAIV

Given the evidence of B cell maturation and antibody secretion in organoid cultures stimulated with LAIV, longitudinal analyses were performed to observe the differentiation process over time. CD38 and CD27 expression patterns on B cells from LAIV-stimulated cultures clearly evolved (FIG. 2A), indicating an early transition to a pre-GC phenotype (CD38+CD27−), and in two of three donors, this was followed by plasmablast differentiation (FIG. 2B). The frequency of plasmablasts typically correlated with the total amount of specific antibodies produced (FIG. 7D). Changes in T cell phenotypes were also observed, including recovery of TFH (CXCR5+CD4+) cells during culture (in both unstimulated and LAIV-stimulated organoids), as well as transient CD4+ and CD8+ T cell activation at day 7 in LAIV-stimulated cultures (FIG. 2C). Organoids prepared from only one of the over 50 donors tested (IMD012) did not produce influenza-specific antibodies (FIG. 2D). With four additional donors, it was confirmed that influenza-specific antibodies are produced with the expected kinetics (between days 5 and 10). The ability of these cultures to produce neutralizing antibodies was gauged; in the two strains tested (of the four LAIV strains), vaccine-stimulated culture supernatants showed neutralizing capabilities, while unstimulated controls did not (FIG. 2E).

The T cell response in HLA-A2+ donors was also assessed using class I tetramers targeting the immunodominant influenza M1 specificity (FIG. 2F). Three of the four donors tested showed a two- to ten-fold expansion of M1-specific CD8+ T cells by day 7 after stimulation with LAIV. The one nonresponder had fewer M1-specific CD8+ T cells in their starting repertoire compared to the other donors, which may explain their poor CD8+ T cell response.

Tetramer staining. HLA-A2 tetramers were prepared as previously described, with ultraviolet (UV)-sensitive peptide cleavage and exchange for the A2 immunodominant influenza M1 peptide (GILGFVFTL) or an irrelevant CMV pp65 peptide (NLVPMVATV) as a control; tetramers were prepared from the peptide-exchanged monomers by conjugation to PE-streptavidin and APC-streptavidin, respectively (eBioscience). For staining, HLA-A2 donor organoids were harvested, washed with FACS buffer and stained for 1 h at 4° C. with tetramers (0.5 μg of monomer per test) in the presence of Fc block. During the last 30 min of tetramer staining, lineage-defining antibodies were added, and samples were washed with FACS buffer. For analysis, influenza M1 tetramer-positive CD8+ T cells were identified as single-positive T cells for the influenza tetramer (negative for CMV tetramer) and with staining above a no-influenza-tetramer staining control.

### Example 3: Spatial Organization of Tonsil Organoids

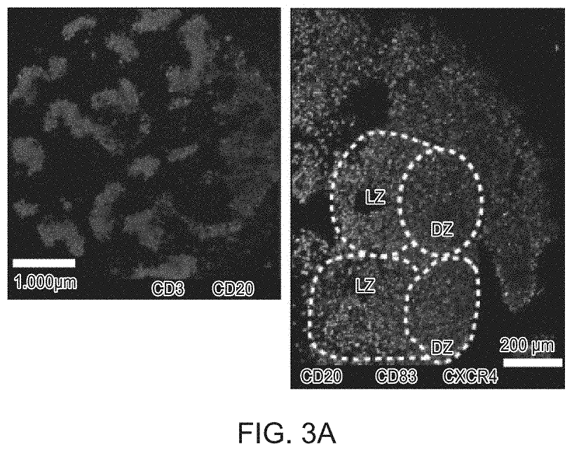

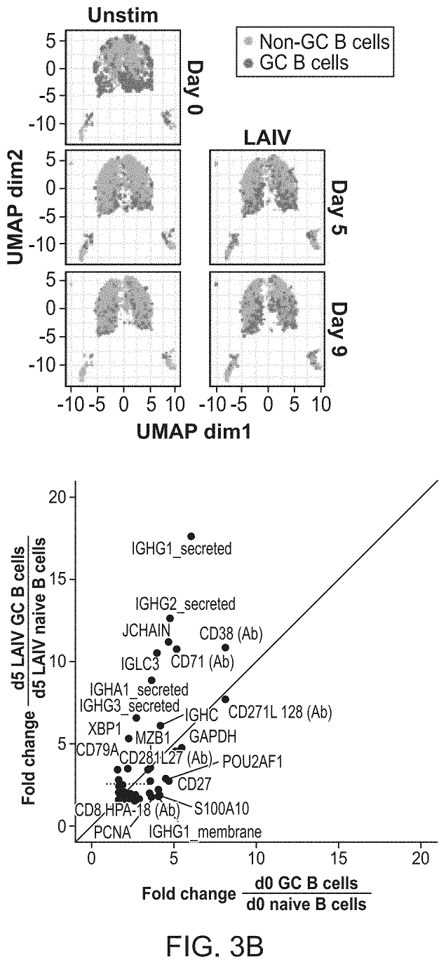

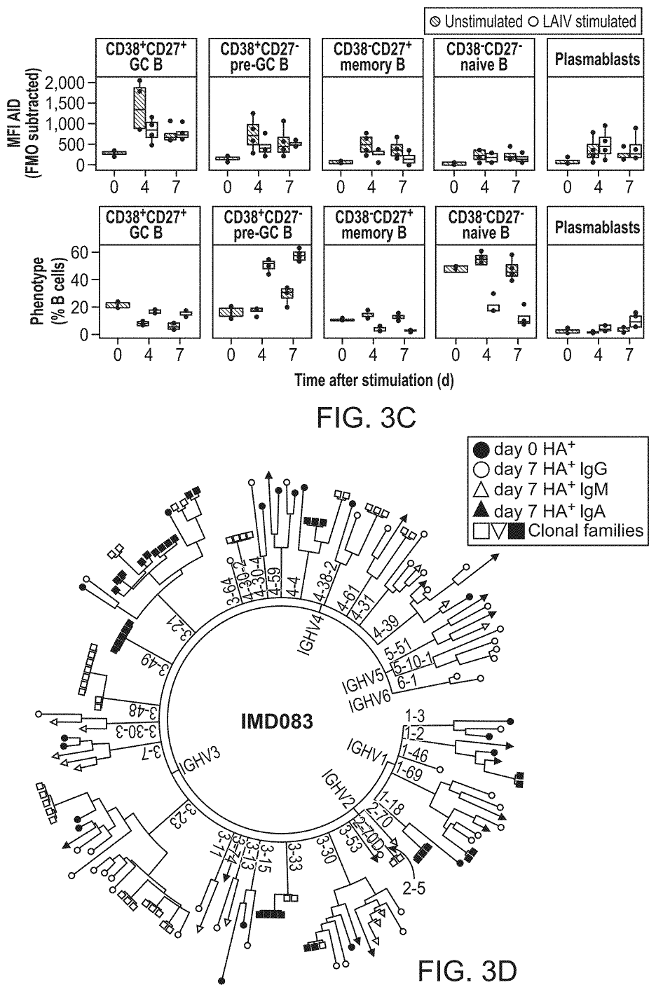

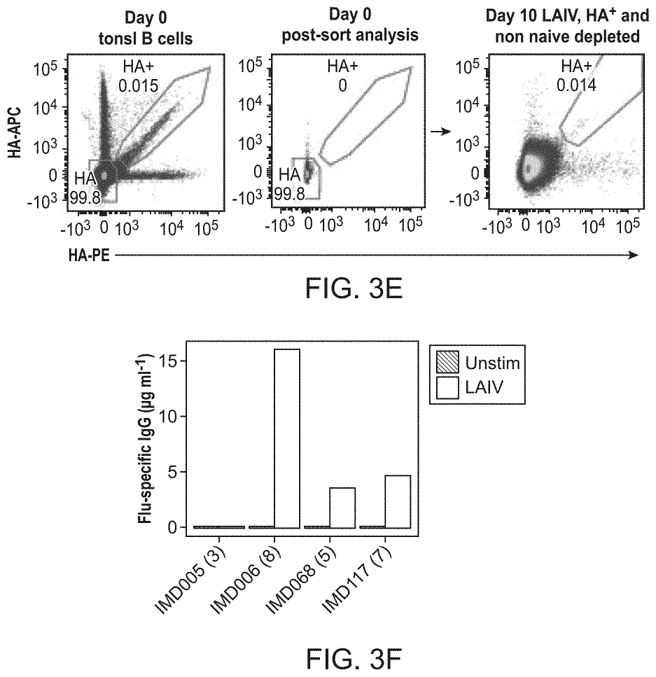

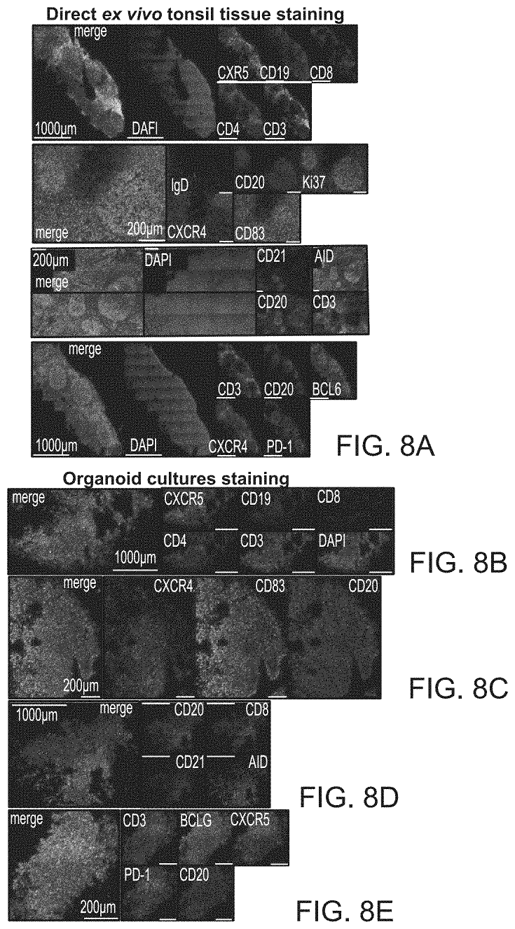

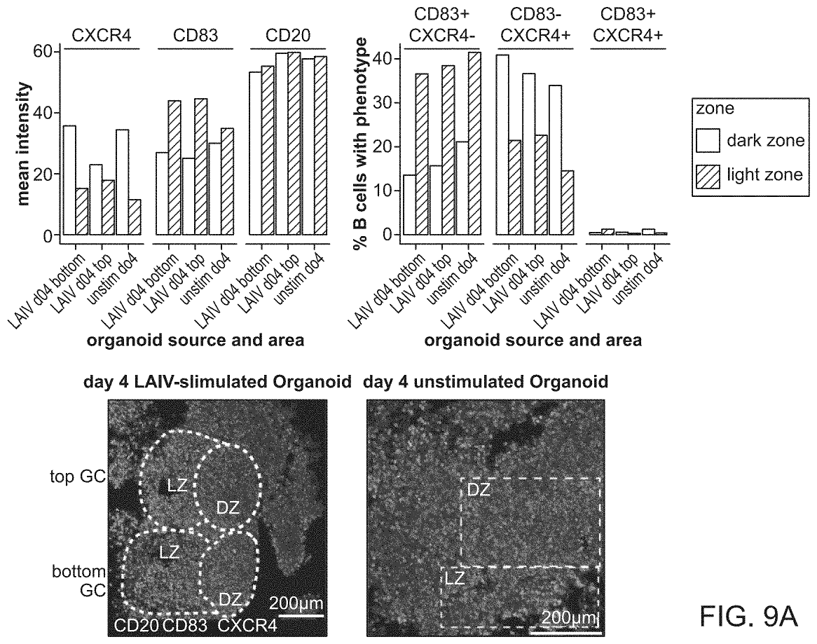

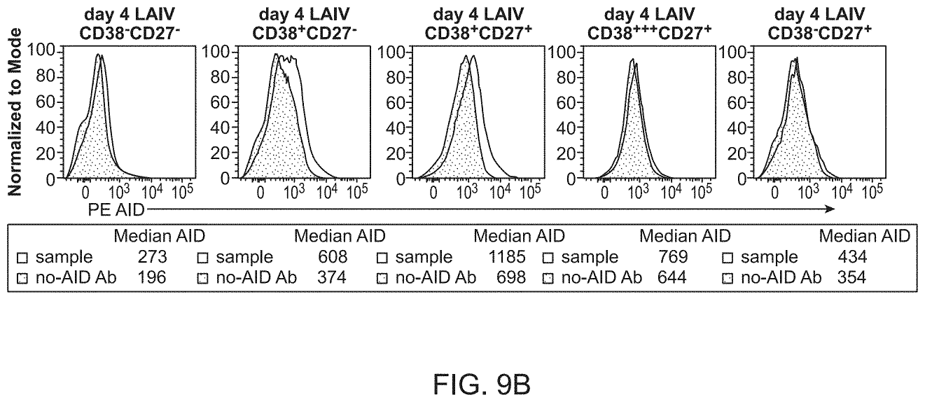

During an adaptive immune response, peripheral lymphoid organs like the tonsils, lymph nodes and spleen develop GCs. B cells congregate in the GC area, whereas T cells are largely in outlying areas In the organoid cultures, GC-like structures with distinct B cell- and T cell-rich aggregates in both LAIV-stimulated and unstimulated cultures were found, starting around 48 h after culture initiation. These progressed to well-defined clusters by days 4-7, particularly in LAIV-treated cultures (FIG. 3A and FIG. 8A-8E). The embedded, frozen sections of three donors by fluorescent confocal microscopy were analyzed and evidence of light and dark zone organization was found, as detected by separation of CD83 and CXCR4 staining B cells, respectively, which are characteristic of GCs (FIG. 3A; representative images FIGS. 8A-8E and 9A).

Immunofluorescence. Immunofluorescence microscopy samples were prepared from frozen tonsil cells stimulated with LAIV and harvested 4 or 7 d after stimulation. Permeable membrane inserts containing organoids were gently immersed in PBS, fixed with 4% paraformaldehyde in PBS for 30 min at 4° C. and washed three times with water. Cultures were kept at room temperature and incubated for 20 min in increasing concentrations of warmed (37° C.) OCT Compound (Fisher) diluted in PBS at 25%, 50% and 75% (vol/vol), with a final incubation in pure OCT. Samples were snap frozen on dry ice, inserts were removed with forceps, and samples were embedded in an additional layer of OCT and frozen. Embedded samples were sectioned at 25 μm and adhered to poly-l-lysine-coated coverslips. Sections were dried for 4 min in a dehumidified chamber and permeabilized in acetone for 10 min at room temperature. Rehydration was performed in stain buffer (1% BSA, 1% normal goat serum and 0.01% sodium azide in PBS) for 30 min. Sections were stained first with primary unconjugated antibodies at room temperature for 3 h. Secondary antibody staining was performed for 1 h at room temperature followed by primary direct conjugate antibodies for 3 h at room temperature.

Primary antibodies. Immunofluorescence staining was performed using the following antibodies at the stated concentrations: BioLegend: CD3-BV421 (SK7; 1:25), CD4-AF594 (RPA-T4; 1:50), CD19-AF647 (SJ25C1; 1:25), CXCR4 (12G5; 1:100), CD21-FITC (Bu32; 1:100), PD-1-AF488 (NAT105; 1:100), BCL-6 (IG191E/A8; 1:50); Sigma-Aldrich: CXCR5 (Polyclonal; 1:100), CD83 (Polyclonal; 1:100); Thermo Fisher: AID (ZA001; 1:100), CD8-AF488 (AMC908; 1:100), CD20-eFluor 615 (L26; 1:100), CD20-eFluor 660 (L26; 1:100); BD Biosciences: Ki67-BV421 (B56; 1:5); Fisher Science: IgD-AF488 (IgD26; 1:10).

Secondary antibodies. The following secondary antibodies from Thermo Fisher and their dilutions were used: goat anti-mouse IgG (H+L) AF Plus 555 (1:200), goat anti-rabbit IgG (H+L) AF Plus 555 (1:200), goat anti-rabbit IgG (H+L) AF Plus 594 (1:200).

After collecting imaging data from antibody markers, slides were stained with 1 μg ml-1 DAPI to visualize nuclear staining. Imaging was performed on an Inverted Zeiss LSM 880 confocal instrument using ×25 magnification. Sample z-stacks were taken at 2-μm slices. DAPI was stimulated at 405 nm, FITC/Alexa Fluor 488 at 488 nm, Alexa Fluor 555 at 561 nm and Alexa Fluor 594 at 594 nm. Images were processed in ImageJ (version 2.0.0). Tile stitching was performed with the Grid/Collection stitching tool using positions from files in the order defined by the image metadata. The fusion method used was Linear Blending, with Computer Overlap and Ignore Calibration (all other parameters were set to default). After despeckling, z-stacks were merged using ZProjection by Maximum Intensity and contrast adjusted to better present structure, and channels were stacked to RGB and a scale bar added.

To quantify the fraction of CXCR4- and CD83-expressing cells in GC areas, GCs were cropped into two portions that resembled light and dark zone-like regions as shown on the imaging figure. CD20-, CD83- and CXCR4-positive cells were manually counted (multipoint tool in ImageJ) in addition to double- and triple-positive cells. CD83+CXCR4−CD20+ and CD83−CXCR4+CD20+ cells in each region were calculated and presented as a proportion of the total CD20+ population from the same area. To determine the mean intensities of marker expressions in light and dark zone regions, images were converted to RGB using ‘stack to RGB’ and then ‘color histogram’ was performed on each channel using ImageJ, which displays a histogram of the intensity values. The mean of each channel was used to compare the average marker expression between different regions of equivalent size.

ELISpot. ASCs were detected using an ELISpot protocol. Cultures that were either stimulated for 7 d with LAIV or left unstimulated were resuspended and enumerated, then plated on inactivated influenza vaccine-coated and blocked 96-well PVDF membrane plates (Millipore). Each sample was plated with three, threefold dilutions in triplicate, and total live-cell counts ranged from 2.22×10⁴ to 1.07×10⁵ cells per well at a 1:9 dilution, which was used for enumeration of ASCs. Cells were incubated on these membranes, undisturbed for 5 h at 37° C. Plates were then washed and treated with horseradish peroxidase-conjugated anti-IgG/IgA/IgM secondary antibody. After incubation overnight at 4° C., plates were washed and developed with AEC substrate (BD), washed 20 times with water, dried, and spots were enumerated. The frequency of ASCs out of total B cells was determined from B cell flow cytometry data analysis and the direct cell enumeration counts.

### Example 4: Expression Profile Characterization

A single-cell RNA-seq analysis was performed along with DNA-barcoded antibodies using the BD Rhapsody platform to characterize the expression profiles of GC-phenotype B cells in organoid cultures. Dimensionality reduction (uniform manifold approximation and projection (UMAP)) was used to analyze GC B cells across different time points (FIG. 3B). Overall, GC B cells (CD38+CD27+) from the different days and time points clustered together, showing that their expression profiles were similar. The top overexpressed genes in GC B cells (compared to naive B cells) at day 5 of LAIV-stimulated cultures were related to antibody secretion (immunoglobulin heavy chains, light chains and J chain) and key B cell differentiation, proliferation and B cell receptor (BCR) signaling genes (XBP1, POU2AF1, S100A10 and PCNA). These were also the top differentially expressed genes between direct ex vivo GC and naive B cells. These data show that the cultures can functionally respond to antigen-specific stimulation, that light and dark zone phenotype B cells separate into distinct areas (FIG. 3A and FIG. 9A) and that B cells with a GC phenotype have similar transcriptional profiles on day 0 and day 5 after culture (FIG. 3B), which collectively support the conclusion that these cultures can be considered organoids.

Single-cell RNA-seq. Cells from either day-0 tonsils (processed, cryopreserved and thawed) or organoids (from day 5 and day 9 after stimulation with LAIV or left unstimulated) were stained with a mixture of fluorophore-conjugated antibodies to enable sorting for CD45+CD19+CD3− B cells and for sequencing detection of DNA-tagged antibodies (CD20 clone L27, CD19 clone SJ25C1, CD71 clone L01.1, IgM clone G20-127, IgA clone A59, CD161 clone HP-3G10, CD27 clone L128, CD38 clone HIT2, IgD clone IA6-2, IgG clone G18-145, CD83 clone HB15E, CXCR4 clone 12G5, TCR Vg9 clone B3, CD3 UCHT1, CD4 clone SK3, CD8 clone RPA-T8 and CXCR5 clone RF8B2). A cocktail of DNA-tagged antibodies was used to allow manual gating on B cells (CD19+CD3−) and B cell subsets (CD27 and CD38) to identify GC B cells and naive B cells. The sorted cells were tagged with sample barcodes to enable pooling. The cells were loaded and captured from the pooled sample using the BD Rhapsody pipeline following the manufacturer's instructions to prepare the libraries, using the targeted human immune gene panel for amplification. Libraries were sequenced using Illumina Novaseq platforms and the resulting data processed using the Rhapsody analysis pipeline. Using SeqGeq (BD) software, individual samples were debarcoded, and B cell subsets were gated based on DNA-barcoded antibodies for CD3, CD19, CD27 and CD38. Individual populations were then exported along with their gene expression profiles for analyses. UMAP dimensionality reduction was run using the R package ‘umap’ on gene (and not DNA-tagged antibody) expression profiles. For fold change expression analyses, genes that were overexpressed in GC B cells by at least 1.5-fold were plotted.

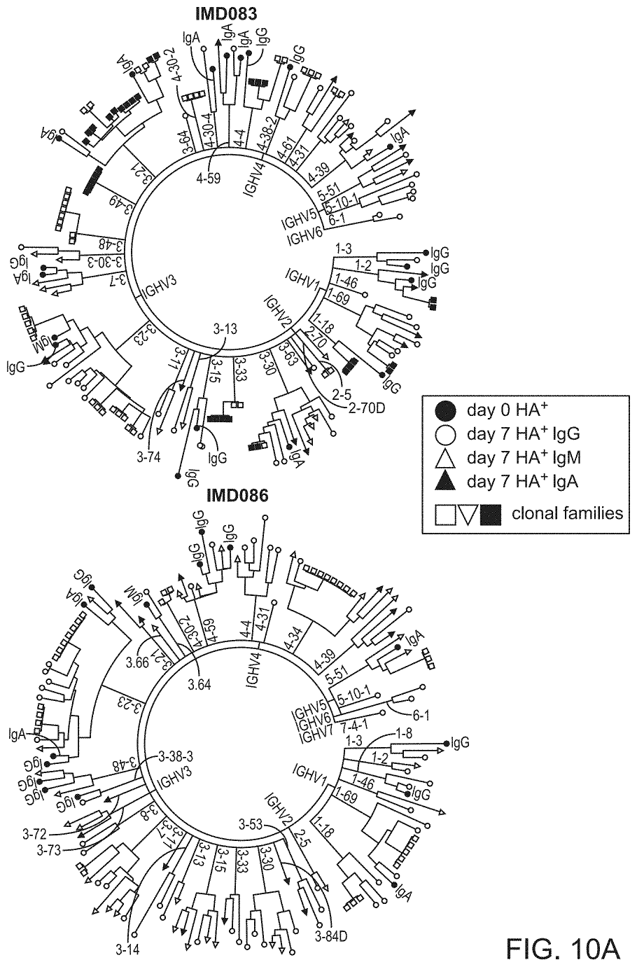

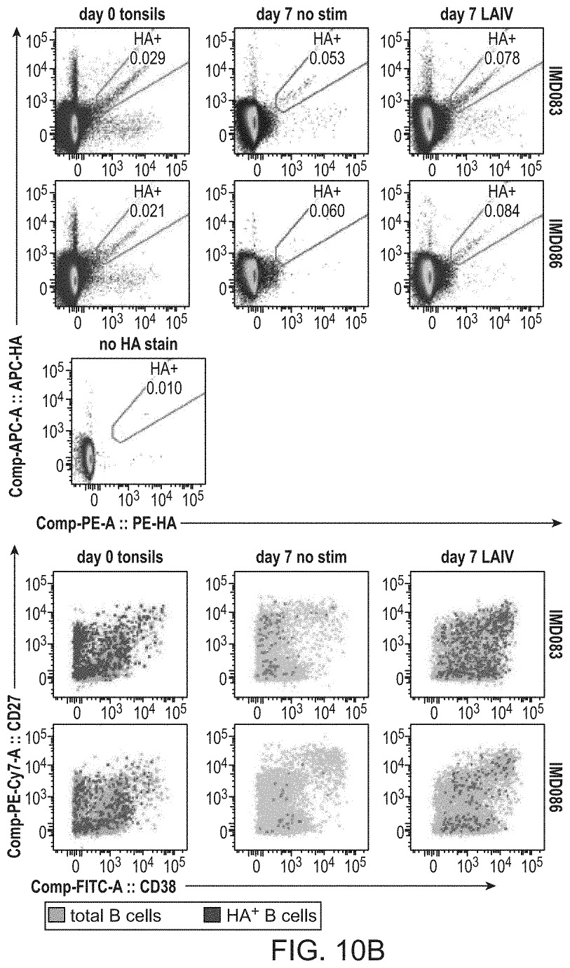

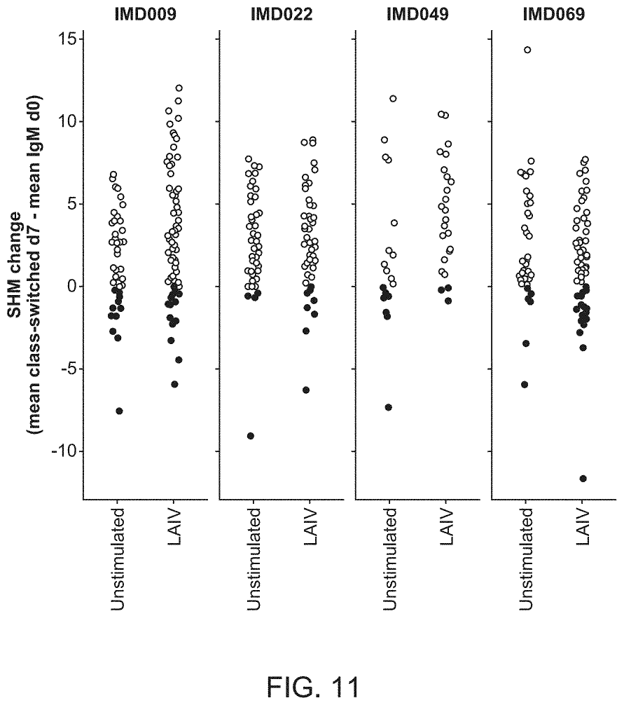

Diversity and maturation of hemagglutinin-specific B cells. Next, it was examined whether activation-induced cytidine deaminase (AID), which is required for both somatic hypermutation and class switching, was expressed in these cultures. AID protein levels (FIG. 3C; see representative staining in FIG. 9B) were significantly increased in pre-GC and GC B cells in 4-day-old organoid cultures (prevalence shown in FIG. 3C). BCRs were then single-cell sequenced from high-affinity A/California 2009 H1N1 hem-agglutinin (HA)-specific plasmablasts (and other activated B cells) using a HA trimer construct on day-0 and day-7 LAIV-stimulated cultures to evaluate affinity maturation and isotype switching. As previously reported in peripheral blood plasmablasts of influenza-vaccinated volunteers, immunoglobulin heavy-chain variable gene cluster (IGHV) 3 and IGHV4 dominated the response (FIG. 3D; FIGS. 10A-10B for complete dataset). HA-specific BCRs from LAIV-stimulated organoids were quite diverse in both gene usage and isotype; numerous oligoclonal expansions were seen at day 7, but these clones were distinct from those detected in day-0 high-affinity HA+ B cells. Clonally related BCR families were also found in LAIV-stimulated organoids, and in one of two donors, clonal families with single amino acid changes were found, showing diversification within HA-specific lineages. Using bulk BCR sequencing of total non-naive B cells, lineages that began as the IgM isotype and class switched during organoid culture (mostly to IgG1 and IgA1) were found, usually with additional mutations, by day 7 (FIG. 11).

B cell receptor sequencing. For isotype-switching analysis, tonsil cells from day-0 or day-7 organoids were harvested, washed with FACS buffer, and stained with a cocktail of lineage-defining antibodies as above in the presence of Fc block, then bulk sorted using a FACS Fusion or Aria II instrument. Bulk sequencing of immunoglobulin heavy-chain gene rearrangements for isotype-switching analysis was carried out as previously reported Briefly, RNA was isolated from sorted (memory CD38−CD27+, GC CD38+CD27+ and plasmablast CD38+++CD27+) cells using Trizol (Thermo Fisher) and reverse transcribed to cDNA using Superscript II (Life Technologies) primed with random hexamer primers. Amplicons from IgM, IgD, IgG, IgA and IgE were PCR amplified in separate reactions, using IGHV framework region 1 primers and isotype primers in the first constant region exon, modified to contain partial Illumina linker sequences, sample barcode sequences and randomized nucleotides to ensure sequence diversity in the initial cycles of sequencing. A second PCR was carried out to complete the Illumina linker sequences before amplicon quantification, pooling, gel extraction (Qiagen) and sequencing on an Illumina MiSeq instrument with 600-cycle kits as 2×300 paired-end reads Bulk BCR heavy-chain sequences were analyzed with an in-house developed pipeline, based on IgBLAST for V, D and J gene segment alignment and CDR-H3 parsing Clonally related sequences in the bulk sequencing data were identified based on shared usage of IGHV and IGHJ genes, equal CDR-H3 length and single-linkage clustering of CDR-H3 nucleotide sequences at a 90% identity threshold.

For analyses of somatic hypermutation and A/California 2009 HA-specific B cells, tonsil cells from day-0 or day-7 organoids were harvested, washed with FACS buffer, then treated with 2 μg per sample (4 μg ml-1) of biotinylated recombinant A/California influenza HA1 hemagglutinin (Y98F mutant; a gift from B. Graham and the Vaccine Research Center) in the presence of Fc block, then washed and stained with 0.2 μg ml-1 of fluorescently labeled streptavidin and a cocktail of lineage-defining antibodies.

For single-cell sorting, HA+ B cells of GC or plasmablast phenotype (CD38+CD27+ or CD38+++CD27+, respectively) were sorted. Single-cell antibody sequencing was performed as previously described. After single-cell sorting into 96-well plates, cDNAs were labeled with well-specific barcode oligonucleotides and pooled by plate, followed by gene-specific PCR and library preparation with previously reported primer sequences. Libraries were sequenced with Illumina MiSeq 2×300 paired-end sequencing. Sequence analysis was performed as previously described. Briefly, fastq generation and plate demultiplexing were completed using the onboard MiSeq Generate FASTQ workflow. After quality filtering, paired reads were stitched, separated by well ID and consensus sequences determined by clustering well ID reads into operational taxonomic units. Consensus operational taxonomic unit sequences were analyzed with version 1.5.7.1 of IMGT HighV-QUEST69. For single-cell data, clonal families were defined by the same V and J gene usage and at least 70% amino acid identity in the CDR3 locus for both heavy and light chains. A caveat of single-cell HA-specific B cell sequencing is that the most vigorously responding B cells may have reduced surface immunoglobulin expression as they convert to antibody secretion, and so it is possible that the highest ASCs could not be captured during HA-specific B cell sorting.

Bulk IgH sequencing and analysis was performed using MIDCIRS as previously described. Sequencing was performed on an Illumina MiSeq with the v3, 600-cycle kit. mRNA molecules were tagged with 12N randomized molecular identifiers (MIDs) during reverse transcription. Reads with the same MID were grouped together and then further clustered into subgroups based on 85% sequence similarity to separate distinct mRNA molecules that were tagged with the same MID. Consensus sequences were then formed from the MID subgroups to average out PCR and sequencing errors and mitigate amplification and sequencing bias. Clonal lineages were defined using single-linkage clustering on the consensus sequences with the same criteria as above. The size of the clonal lineage refers to the total number of consensus sequences, or mRNA molecules, that make up the lineage, while the diversity of the lineage refers to the number of unique consensus sequences within the lineage.

To infer HA specificity of total, unsorted B cells from day-7 tonsil cultures, all FACS-sorted, HA-specific B cell sequences were first grouped together (n=20,977). Then, all nucleotide sequences were translated to amino acid sequences, and each day-7 sequence was aligned to the pool of HA+ sequences to find the minimum distance to the nearest HA-specific sequence. If any sequence within a clonal lineage was found to be an exact match or one substitution away from a known HA-specific sequence, the lineage was labeled ‘HA-inferred’. Clonal lineages were then split into HA-inferred and non-A/California HA for further analysis.

### Example 5: Affinity Maturation in Tonsil Organoids

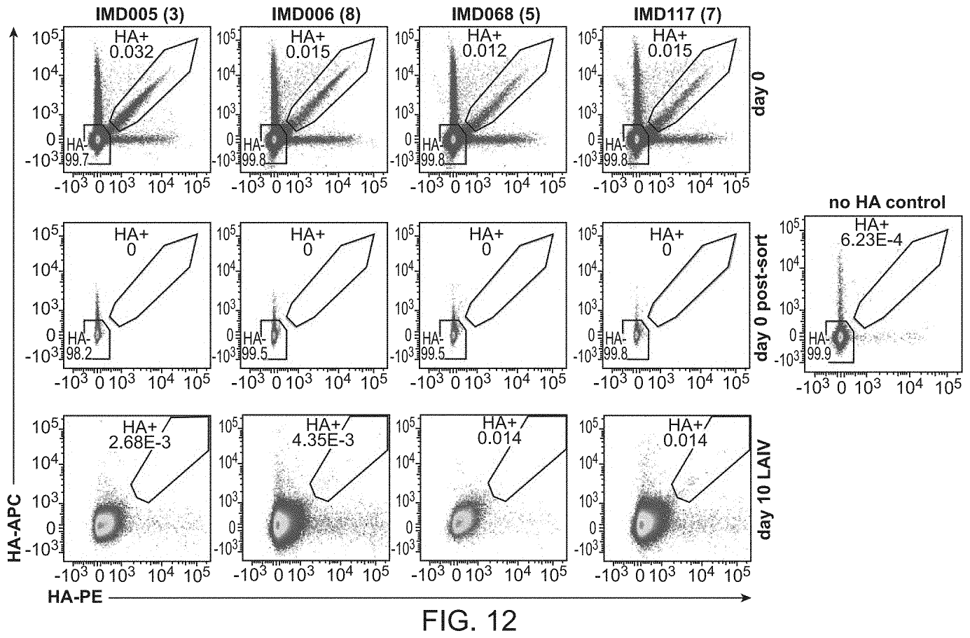

Affinity maturation is another key function of GCs. To evaluate this, depletion experiments using FACS to eliminate preexisting high-affinity HA+ B cells and also any non-naive B cells from the tonsil cell pool were performed. Cultures were then prepared from the depleted cells, stimulated with LAIV, and stained again on day 10 to assess the development of new high-affinity HA+ B cells (FIG. 3E and FIG. 12). In three of four donors tested, HA+ B cells reappeared in depleted cultures and influenza-specific secreted antibodies were detectable (FIG. 3F), demonstrating that affinity maturation can be directly observed in this system.

Cell depletion experiments. Thawed tonsil cells were stained and bulk sorted into culture medium using a FACS Aria II or Fusion (BD). Sorting experiments involved separating individual cell subsets using the following markers: myeloid cells and DCs (CD45+ CD3−CD19−HLA-DR+, CD116+ or CD33+), pDCs (CD45+CD3−CD19−HLA-DR+CD123+), Treg cells (CD45+CD3+CD19−CD4+CD25+CD127lo), total B cells (CD45+CD3−CD19+), non-naive B cells (CD38+ and/or CD27+IgM− and/or IgD−), naive B cells (CD38−CD27−IgM+IgD+), pre-GC B cells (CD38+CD27−), GC B cells (CD38+CD27+), memory B cells (CD38−CD27+) and plasmablasts/plasma cells (CD38+++CD27+). Cell types were depleted by FACS from day-0 tonsil cells and cultured with LAIV for 7 d. As controls, depleted cultures were reconstituted with the cell type that was originally sorted and plated at the same cell density as the depleted cultures. For depletion of HA-specific B cells, tonsil samples were stained as described in ‘BCR sequencing’ and HA-specific B cells were defined as CD45+CD19+CD3−HA+. A post-sort analysis was used to ensure depletion purity.

Statistical analysis. All statistical analyses were performed in R. For comparing paired samples (frequencies of plasmablasts and antibody secretion in unstimulated versus LAIV-stimulated cultures, standard versus Transwell responses, unstimulated versus MMR-stimulated cultures, somatic hypermutation analyses, studying the effect of CD4+ cell depletion on plasmablast differentiation and specific antibody secretion), paired Wilcoxon signed-rank tests (two-sided) were performed. To analyze the CD4 depletion effect in young versus older children, unpaired Mann-Whitney U tests (two-sided) were performed. Welch's t-tests (two-sided) were used to compare the somatic hypermutation levels between culture conditions and the clone size and diversity of inferred HA-specific and nonspecific B cell lineages. Two-sided, paired t-tests were used to compare the effect of PE stimulation with and without adjuvants on the frequency of PE+ B cells.

Next, IgH sequencing was performed to analyze the BCR repertoire in naive versus affinity matured B cells in response to LAIV stimulation. Here, cultures were prepared where naive, HA− B cells were the only B cell source (FIG. 4A). Day-0 HA+ B cells were used as the reference for high-affinity BCRs and to infer HA specificity. Organoid cultures were harvested on day 7 for bulk BCR repertoire sequencing (FIG. 4A). The repertoires of these cultures were compared to both the in vivo high-affinity HA+ B cells from day-0 tonsil cells that were depleted by FACS and the HA− naive B cells that went into culture preparations. Of the six donors tested, five secreted antibodies specific for vaccine components and four had detectable antibodies specific for the A/California 2009 H1N1 HA protein on day 7 after stimulation (FIG. 4B), showing that HA-specific antibodies can be derived from naive B cells.

Somatic hypermutation was significantly increased in day-7 LAIV-stimulated cultures compared to unstimulated control cultures and was particularly enhanced where A/California 2009 H1N1 HA antibodies were detected (IMD006, 013, 014 and 102; FIG. 4C). The development of BCR sequences likely (within one amino acid) to be specific for influenza strain A/California H1N1 were followed. In two of the six donors tested (IMD006 and IMD014), the B cell clones with inferred HA specificity for this strain were also larger than those without known HA specificity and this was dependent on vaccine stimulation (FIG. 4D). In these same donors, the average diversity was also increased, with more unique molecules per clonal lineage in HA-specific B cells from LAIV-stimulated cultures (FIG. 4D). Other expanded and diverse lineages only in the vaccine-stimulated cultures were found, likely responding to other antigens or strains. Direct evidence for BCR sequence evolution to increased HA affinity (FIG. 4E) was found. Here a root sequence with four nucleotide mutations from the germline heavy-chain sequence acquired an additional ten mutations to achieve an exact amino acid match to a known high-affinity HA specificity. Taken together, these data confirm that antigen-driven somatic hypermutation, affinity maturation and class switching are support in tonsil organoid cultures.

### Example 6: Contributions of Individual Cell Types to Influenza Responses

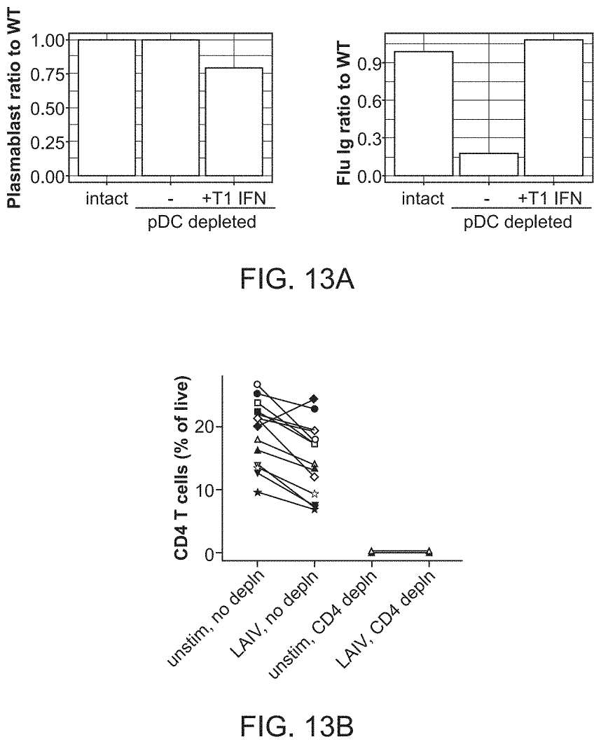

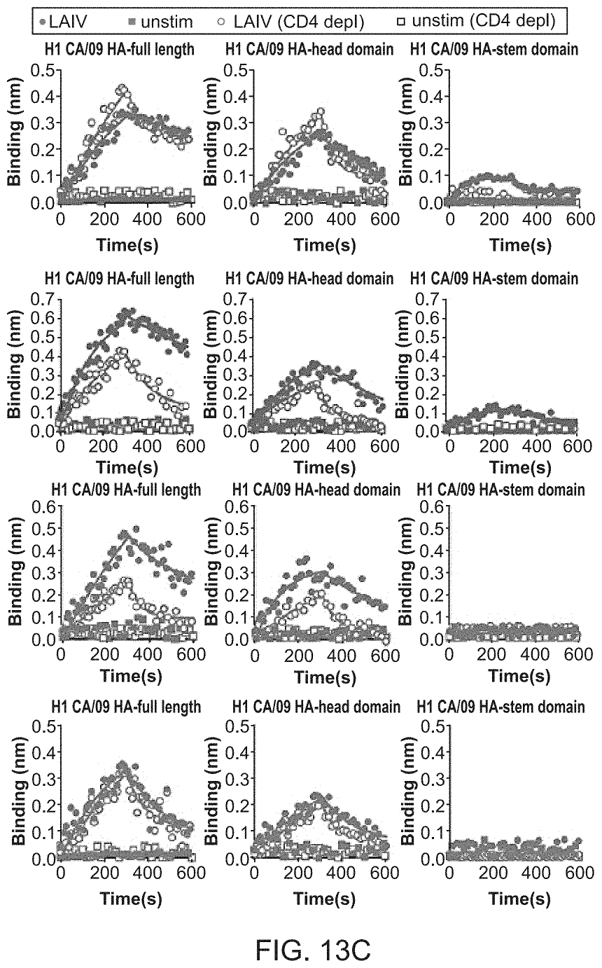

A major advantage of in vitro systems is the ability to define the essential components. Therefore, APC, T cell and B cell subsets were depleted and plasmablast differentiation was compared (FIGS. 5B and 5D) to wild type organoids. Despite their low starting frequency (0.1-0.5% of live), depleting myeloid and plasmacytoid dendritic cell (pDC) populations completely abrogated plasmablast differentiation (FIG. 5A) and depleting pDCs alone was sufficient to dramatically reduce the antibody response to LAIV (FIG. 5B). The effect of pDC loss was rescued by adding type I interferon (IFN), showing that type I IFN production is the main pDC function (FIG. 13A).

As expected, depleting CD4+ cells (FIG. 13B) significantly reduced plasmablast differentiation, although some influenza-specific antibodies were still detectable (FIGS. 5C-5D). The effect of donor age was analyzed and it was found that CD4+ T cell-depleted organoids from younger children were almost entirely unable to secrete influenza-specific antibody (FIG. 5C), in contrast to older donors who could make antibody responses.

The affinities of antibodies were characterized against A/California/07/2009 H1N1 HA in CD4+-depleted cultures. The dissociation rates for antibodies raised in the absence of CD4+ cells were four- to ten-fold higher than wild-type controls (FIG. 5F and FIG. 13C), even when the quantity of specific antibody was comparable between intact and CD4+-depleted cultures. Thus, CD4+ T cells are important for both the magnitude and affinity of the antibody response.

It was determined that three to four cell types were minimally required to consistently achieve detectable plasmablast differentiation and naive antibody responses against LAIV: naive B cells, APCs, CD4+ T cells and, in some donors, a mixed population of CD45− stromal cells (FIG. 5G). Notably, naive CD4+ T cells were either as good or better than memory CD4+ T cells in their ability to help B cells differentiate into plasmablasts and stimulate influenza-specific antibodies (FIG. 5H).

### Example 7: Response to Other Antigens and the Effect of Adjuvants

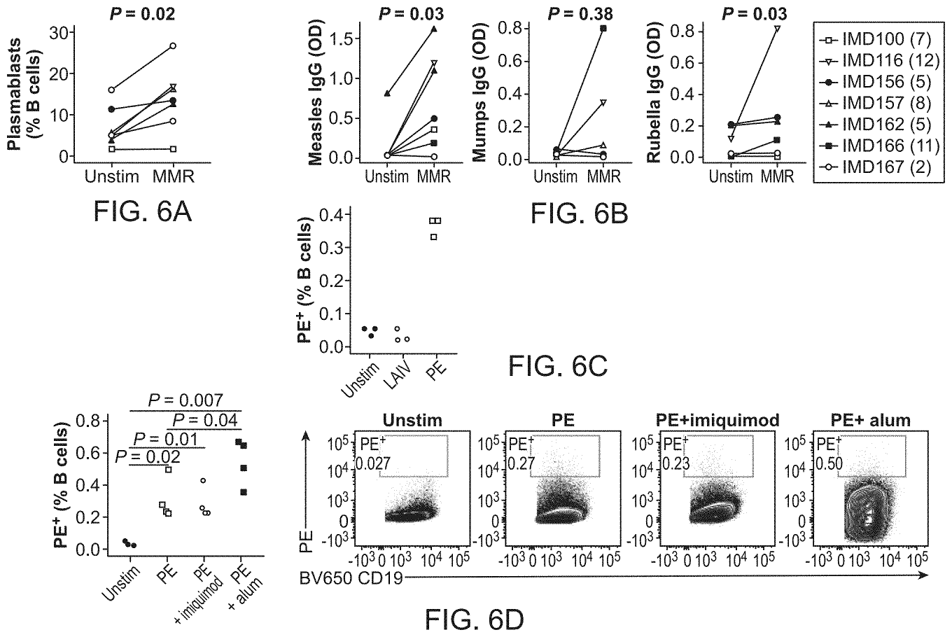

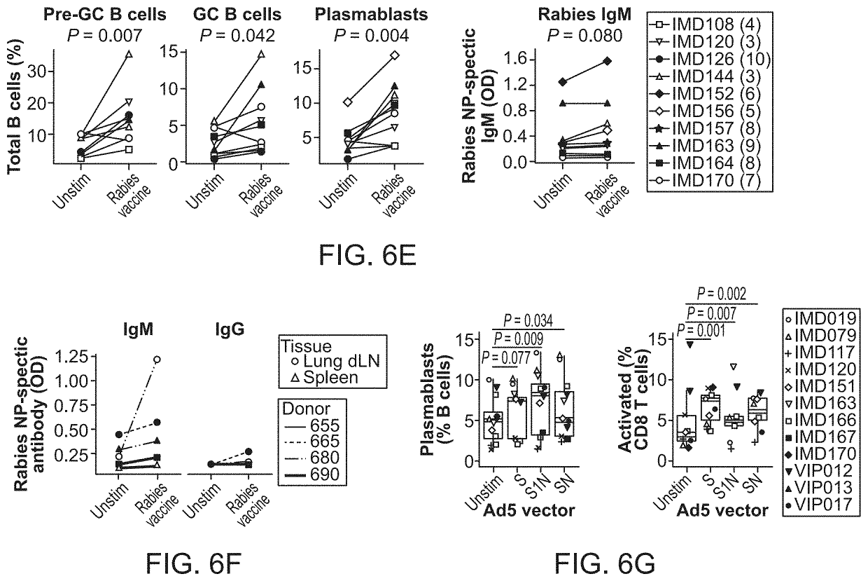

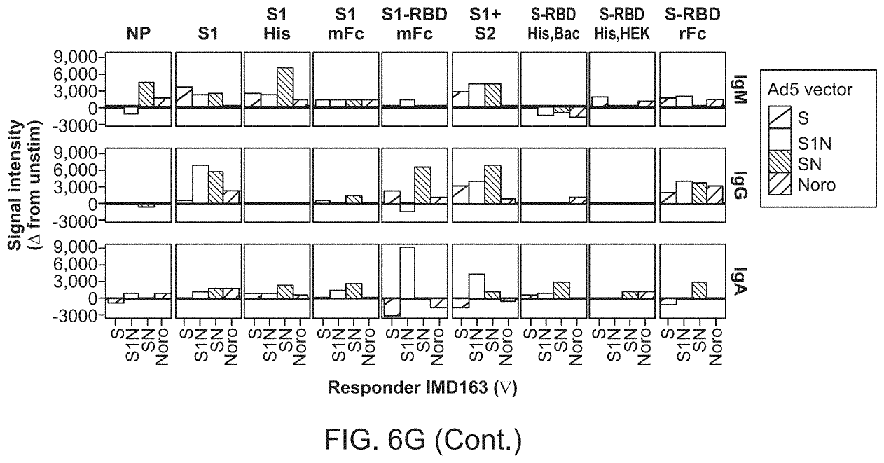

It was then investigated whether tonsil organoids could also respond to non-influenza memory antigens. Tonsil organoids were stimulated with the measles, mumps and rubella (MMR) vaccine, which is recommended to be administered at 12 months, and assessed plasmablast formation and antigen-specific IgG responses. These cultures had significantly increased plasmablasts compared to controls (FIG. 6A), and six of the seven donors made measles-specific IgG, while the mumps and rubella responses were weaker and only detected in three of the older children tested (FIG. 6B).

Affinity binding experiments. Binding affinities of the indicated antigens to antibody-containing supernatant samples were determined by biolayer interferometry using an Octet QK instrument (Pall ForteBio). For analysis of the effects of T cells on antibody responses, antibodies were purified from culture supernatants using a Protein G affinity column (GE Healthcare Life Sciences). The full-length, head and stem domain H1 CA/09 HA antigens were purified as described previously using a Ni-NTA affinity column followed by size-exclusion cleanup. The purified antigens were captured on anti-penta-his (HIS1K) biosensor tips in PBS-T (PBS with 0.05% Tween 20 (pH 7.4)). The ligand-bound sensors were dipped into control wells or purified antibodies (200-500 nM). A similar antibody concentration was used for all the evaluated conditions from an individual donor. Unliganded sensors dipped into the analyte served as controls for nonspecific binding. The traces were processed using ForteBio Data Analysis Software (v8.0). The data were fitted globally to a simple 1:1 Langmuir interaction model to obtain the kinetic parameters. Each binding interaction was repeated at least thrice.

To determine whether tonsil organoids could serve as a platform for priming antigen-specific adaptive responses, cells were first stimulated with naive antigen phycoerythrin (PE), with and without adjuvants. Here a substantial (tenfold) increase in PE+ B cells in PE-stimulated cultures was seen as compared to unstimulated or irrelevant antigen-stimulated cultures (FIG. 6C). The frequency of PE+ B cells could also be enhanced by alum hydrogel but not imiquimod (FIG. 6D), indicating that at least some naive responses can be elicited and modulated by appropriate adjuvants. The ability of tonsils, lung-draining lymph nodes and spleen cultures to respond to the T cell-dependent rabies vaccine was then tested. The donors had not previously received this vaccine. Tonsil B cells were more differentiated in vaccine-stimulated cultures after 14 d in organoid culture and four of ten donors had detectable increases in rabies nucleoprotein-specific IgM (FIG. 6E). Lung-draining lymph node and spleen organoids also showed modest rabies-specific antibody production, with some specific IgM but not IgG production, consistent with a priming response (FIG. 6F).

### Example 8: Testing Vaccine Candidates on Tonsil Organoids

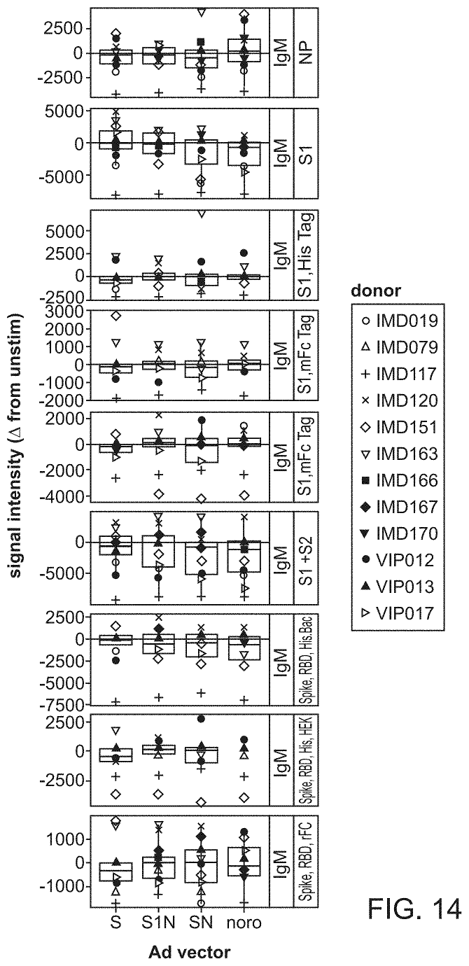

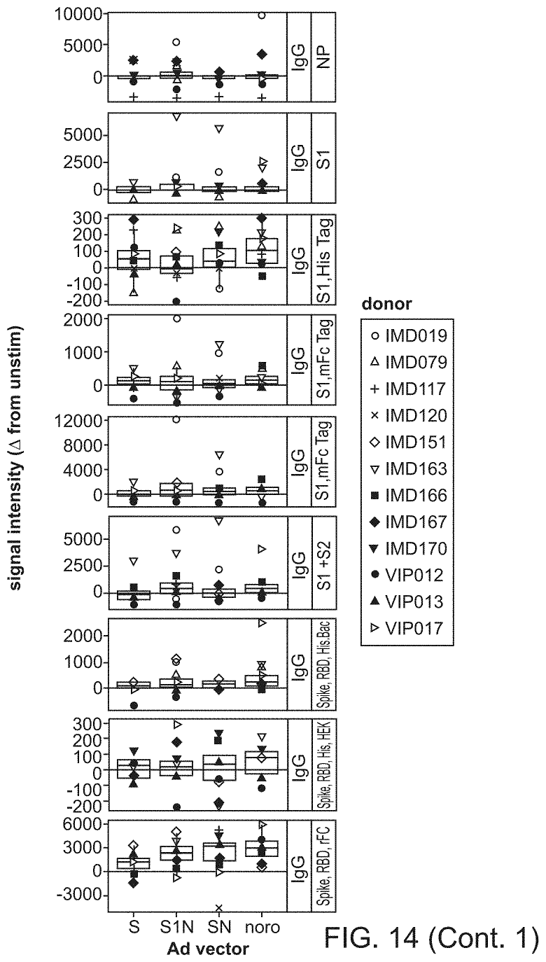

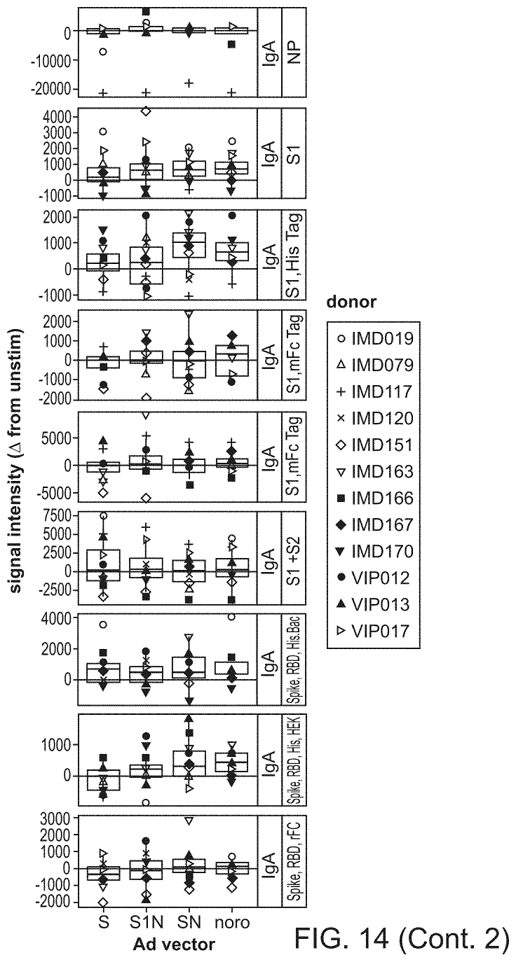

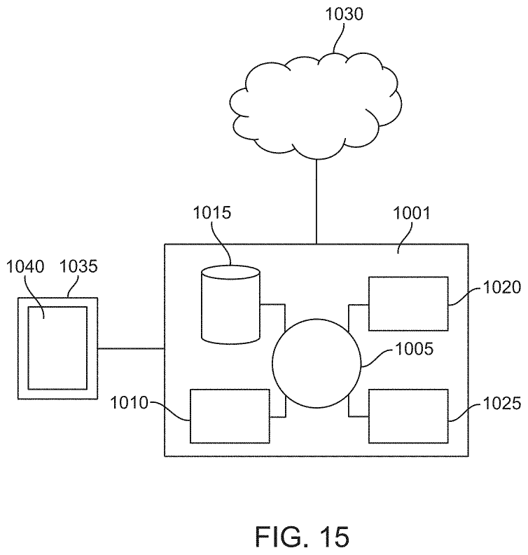

The ability of tonsil organoids to respond to a series of vaccine candidates developed in response to severe acute respiratory syndrome coronavirus 2 (SARS-CoV-2) was investigated. These vaccine candidates use a replication-deficient type 5 adenovirus (Ad5) that encodes either the full-length viral spike protein, spike and nucleocapsid protein or the S1 spike subunit with nucleocapsid in the Ad5 E1 region. All tonsil tissue samples were collected before the SARS-CoV-2 pandemic and thus naive to these antigens. On day 14 after stimulation, plasmablast differentiation in a subset of donors and significant CD8 T cell activation was observed compared to unstimulated controls (FIG. 6G). Using a protein microarray to detect antibody specificities against SARS-CoV-2, specific IgG and IgA antibodies were present for some spike and nucleocapsid proteins in a few donors. One particularly robust responder, IMD163, is shown in FIG. 6G (data from all donors are available in FIG. 14). This shows that immune organoid cultures can be used to analyze and compare vaccine candidates to new and previously encountered antigens

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An in-vitro cell cluster comprising lymphoid cells,
wherein said in-vitro cell cluster is derived from lymphoid tissue, said in-vitro cell cluster comprising a germinal center and an aggregate of T-cells; wherein said in-vitro cell cluster is configured to maintain said germinal center and said aggregate of T-cells and cellular respiration for at least 24 hours.

2. The in-vitro cell cluster of claim 1, further comprising one or more adjuvants.

3. (canceled)

4. The in-vitro cell cluster of claim 1, wherein said germinal center is configured to perform one or more of: hypermutation maturation, affinity maturation, plasmablast differentiation, class switching recombination, and antigen-specific antibody production.

5. The in-vitro cell cluster of claim 1, wherein cells of said in-vitro cell cluster are configured to differentiate to form said in-vitro cell cluster upon exposure to an antigen.

6. The in-vitro cell cluster of claim 5, wherein said antigen is a peptide, protein or fragment thereof.

7. The in-vitro cell cluster of claim 6, wherein said protein is a viral protein, a growth factor, a cancer related protein, or an auto-immune disease related protein.

8. The in-vitro cell cluster of claim 1, wherein said lymphoid cells are derived from tonsil tissue, spleen tissue, adenoid tissue, thymus tissue, or lymph node tissue.

9. The in-vitro cell cluster of claim 1, wherein said germinal center comprises antigen presenting cells (APCs).

10. The in-vitro cell cluster of claim 9, wherein said APCs comprise B-cells or dendritic cells.

11. The in-vitro cell cluster of claim 10, wherein said dendritic cells are follicular dendritic cells.

12. The in-vitro cell cluster of claim 10, wherein said B-cells comprise CD38+ B-cells and/or CD27+ B-cells.

13. (canceled)

14. The in-vitro cell cluster of claim 9, wherein said T-cells comprise CD8+ T-cells and/or CD4+ T-cells.

15-35. (canceled)

36. A method for generating antibodies from a lymphoid organoid, comprising:
a. placing cells comprising lymphoid cells in a media to produce said lymphoid organoid, wherein said lymphoid organoid comprises a germinal center and an aggregate of T-cells;
b. introducing an antigen to said media;
c. incubating said lymphoid organoid with said antigen to generate said antibodies; and
d. isolating said antibodies from said lymphoid organoid.

37. The method of claim 36, wherein (a) further comprises introducing a B-cell activating factor to said media.

38. The method of claim 36, wherein (b) further comprises introducing an adjuvant to said media.

39. (canceled)

40. The method of claim 38, wherein (c) further comprises incubating said lymphoid organoid with said antigen and said adjuvant.

41. The method of claim 38, wherein said adjuvant is introduced after said antigen.

42. (canceled)

43. The method of claim 36, further comprises obtaining said cells from a subject wherein said lymphoid cells are derived from tonsil tissue, spleen tissue, adenoid tissue, thymus tissue, or lymph node tissue.

44. (canceled)

45. (canceled)

46. The method of claim 36, wherein said antigen is a peptide, protein, or fragment thereof.

47. The method of claim 46, wherein said protein is a viral protein, a growth factor, a cancer related protein, or an auto-immune disease related protein, and wherein said viral protein is derived from a coronavirus or a flu virus.

48.-51. (canceled)
